(19) United States
(12) Patent Application Publication
(10) Pub. No.: US 2018/0163229 A1
(43) Pub. Date: Jun. 14, 2018

(54) NOVEL RECOMBINANT ADENO-ASSOCIATED VIRUS CAPSIDS CONTAINING A DESIGNED ANKYRIN REPEAT PROTEIN (DARPIN) OR FRAGMENT THEREOF

(71) Applicant: The Board of Trustees of the Leland Stanford Junior University, Palo Alto, CA (US)

(72) Inventors: Mark A. Kay, Los Altos, CA (US); Robert C. Muench, Palo Alto, CA (US)

(21) Appl. No.: 15/838,222

(22) Filed: Dec. 11, 2017

Related U.S. Application Data
(60) Provisional application No. 62/433,170, filed on Dec. 12, 2016.

(51) Int. Cl. C12N 15/861; C07K 14/075; A61K 35/761; A61K 39/235

(52) U.S. Cl. CPC C12N 15/861; C07K 14/075; A61K 35/761; C07K 2319/735; C12N 2710/10045; C12N 2710/10034; A61K 39/235

(57) ABSTRACT

The present invention relates to variant AAV capsid polypeptides containing designed ankyrin repeat proteins (DARPins), wherein the variant capsid polypeptides exhibit an enhanced neutralization profile, increased transduction, and/or tropism in human liver and/or hepatocyte cells, or human pancreas and/or pancreatic cells, as compared to capsid polypeptides that do not include DARPins.

### CROSS REFERENCE TO RELATED APPLICATIONS

This application claims the benefit of U.S. Provisional Application No. 62/433,170 filed Dec. 12, 2016, which is expressly incorporated herein by reference in its entirety.

### STATEMENT AS TO RIGHTS TO INVENTION MADE UNDER FEDERALLY SPONSORED RESEARCH OR DEVELOPMENT

This invention was made with Government support under contracts AI116698 and DK089569 awarded by the National Institutes of Health. The Government has certain rights in the invention.

### REFERENCE TO A “SEQUENCE LISTING”, A TABLE, OR A COMPUTER PROGRAM, LISTING APPENDIX SUBMITTED ON A COMPACT DISK

This invention incorporated by reference the Sequence Listing text copy submitted herewith, which was created on Dec. 11, 2017, entitled 068597_5033 US_SequenceListing_ST25.txt which is 36.0 kilobytes in size.

### BACKGROUND OF THE INVENTION

Genetic disorders caused by absence of or a defect in a desirable gene (loss of function) or expression of an undesirable or defective gene (gain of function) lead to a variety of diseases. At present, adeno-associated virus (AAV) vectors are recognized as the gene transfer vectors of choice for therapeutic applications since they have the best safety and efficacy profile for the delivery of genes in vivo. Of the AAV serotypes isolated so far, AAV2 and AAV8 have been used to target the liver of humans affected by severe hemophilia B. Both vectors worked efficiently and, in the case of AAV8, long-term expression of the therapeutic transgene was documented. Recent data from humans showed that targeting the liver with an AAV vector achieves long-term expression of the FIX transgene at therapeutic levels. Additionally, several Phase 1 and Phase 2 clinical trials using AAV serotypes 1, 2 and chimeric 2.5 have been reported for the treatment of Duchenne muscular dystrophy (DMD) and alpha-1 antitrypsin deficiency (D. E. Bowles, S. W J McPhee, C. Li, S. J. Gray, J. J. Samulski, A. S. Camp, J. Li, B. Wang, P. E. Monahan, J. E. Rabinowitz, J. C. Grieger, La. Govindasamy, M. Agbandje-McKenna, X. Xiao and R. J. Samulski, Molecular Therapy, 20, 443-455 (2012); M. L. Brantly, J. D. Chulay, L. Wang, C. Mueller, M. Humphries, L. T. Spencer, F. Rouhani, T. J. Conlon, R. Calcedo, M. R. Betts, C. Spencer, B. J. Byrne, J. M. Wilson, T. R. Flotte, Sustained transgene expression despite T lymphocyte responses in a clinical trial of rAAV1-AAT gene therapy. Proceedings of the National Academy of Sciences of the United States of America 106, 16363-16368 (2009); T. R. Flotte, M. L. Brantly, L. T. Spencer, B. J. Byrne, C. T. Spencer, D. J. Baker, M. Humphries, Phase I trial of intramuscular injection of a recombinant adeno-associated virus alpha 1-antitrypsin (rAAV2-CB-hAAT) gene vector to AAT-deficient adults. Human gene therapy 15, 93-128 (2004); T. R. Flotte, B. C. Trapnell, M. Humphries, B. Carey, R. Calcedo, F. Rouhani, M. Campbell-Thompson, A. T. Yachnis, R. A. Sandhaus, N. G. McElvaney, C. Mueller, L. M. Messina, J. M. Wilson, M. Brantly, D. R. Knop, G. J. Ye, J. D. Chulay, Phase 2 clinical trial of a recombinant adeno-associated viral vector expressing alpha1-antitrypsin: interim results. Human gene therapy 22, 1239-1247 (2011); C. Mueller, J. D. Chulay, B. C. Trapnell, M. Humphries, B. Carey, R. A. Sandhaus, N. G. McElvaney, L. Messina, Q. Tang, F. N. Rouhani, M. Campbell-Thompson, A. D. Fu, A. Yachnis, D. R. Knop, G. J. Ye, M. Brantly, R. Calcedo, S. Somanathan, L. P. Richman, R. H. Vonderheide, M. A. Hulme, T. M. Brusko, J. M. Wilson, T. R. Flotte, Human Treg responses allow sustained recombinant adeno-associated virus-mediated transgene expression. The Journal of clinical investigation 123, 5310-5318 (2013)).

Adeno-associated virus (AAV), a member of the Parvovirus family, is a small nonenveloped, icosahedral virus with single-stranded linear DNA genomes of 4.7 kilobases (kb). AAV is assigned to the genus, Dependovirus, because the virus was discovered as a contaminant in purified adenovirus stocks (D. M. Knipe, P. M. Howley, Field's Virology. Lippincott Williams & Wilkins, Philadelphia, ed. Sixth, 2013). In its wild-type state, AAV depends on a helper virus—typically adenovirus—to provide necessary protein factors for replication, as AAV is naturally replication-defective. The 4.7-kb genome of AAV is flanked by two inverted terminal repeats (ITRs) that fold into a hairpin shape important for replication. Being naturally replication-defective and capable of transducing nearly every cell type in the human body, AAV represents an ideal vector for therapeutic use in gene therapy or vaccine delivery. In its wild-type state, AAV's life cycle includes a latent phase during which AAV genomes, after infection, are site specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains (A. Vasileva, R. Jessberger, Nature reviews. Microbiology, 3, 837-847 (2005)). Rep and Cap are then replaced with an array of possible transfer vector configurations to perform gene addition or gene targeting. These vectorized recombinant AAVs (rAAV) transduce both dividing and non-dividing cells, and show robust stable expression in quiescent tissues. The number of rAAV gene therapy clinical trials that have been completed or are ongoing to treat various inherited or acquired diseases is increasing dramatically as rAAV-based therapies increase in popularity. Similarly, in the clinical vaccine space, there have been numerous recent preclinical studies and one ongoing clinical trial using rAAV as a vector to deliver antibody expression cassettes in passive vaccine approaches for human/simian immunodeficiency virus (HIV/SIV), influenza virus, henipavirus, and human papilloma virus (HPV). (See, P. R. Johnson, B. C. Schnepp, J. Zhang, M. J. Connell, S. M. Greene, E. Yuste, R. C. Desrosiers, K. R. Clark, Nature medicine 15, 901-906 (2009); A. B. Balazs, J. Chen, C. M. Hong, D. S. Rao, L. Yang, D. Baltimore, Nature 481, 81-84 (2012); A. B. Balazs, Y. Ouyang, C. M. Hong, J. Chen, S. M. Nguyen, D. S. Rao, D. S. An, D. Baltimore, Nature medicine 20, 296-300 (2014); A. B. Balazs, J. D. Bloom, C. M. Hong, D. S. Rao, D. Baltimore, Nature biotechnology 31, 647-652 (2013); M. P. Limberis, V. S. Adam, G. Wong, J. Gren, D. Kobasa, T. M. Ross, G. P. Kobinger, A. Tretiakova, J. M., Science translational medicine 5, 187ra172 (2013); M. P. Limberis, T. Racine, D. Kobasa, Y. Li, G. F. Gao, G. Kobinger, J. M. Wilson, Vectored expression of the broadly neutralizing antibody FI6 in mouse airway provides partial protection against a new avian influenza A virus, H7N9. Clinical and vaccine immunology: CVI 20, 1836-1837 (2013); J. Lin, R. Calcedo, L. H. Vandenberghe, P. Bell, S. Somanathan, J. M. Wilson, Journal of virology 83, 12738-12750 (2009); I. Sipo, M. Knauf, H. Fechner, W. Poller, O. Planz, R. Kurth, S. Norley, Vaccine 29, 1690-1699 (2011); A. Ploquin, J. Szecsi, C. Mathieu, V. Guillaume, V. Barateau, K. C. Ong, K. T. Wong, F. L. Cosset, B. Horvat, A. Salvetti, The Journal of infectious diseases 207, 469-478 (2013); D. Kuck, T. Lau, B. Leuchs, A. Kern, M. Muller, L. Gissmann, J. A. Kleinschmidt, Journal of virology 80, 2621-2630 (2006); K. Nieto, A. Kern, B. Leuchs, L. Gissmann, M. Muller, J. A. Kleinschmidt, Antiviral therapy 14, 1125-1137 (2009); K. Nieto, C. Stahl-Hennig, B. Leuchs, M. Muller, L. Gissmann, J. A. Kleinschmidt, Human gene therapy 23, 733-741 (2012); and L. Zhou, T. Zhu, X. Ye, L. Yang, B. Wang, X. Liang, L. Lu, Y. P. Tsao, S. L. Chen, J. Li, X. Xiao, Human gene therapy 21, 109-119 (2010).) The properties of non-pathogenicity, broad host range of infectivity, including non-dividing cells, and potential site-specific chromosomal integration make AAV an attractive tool for gene transfer.

The first rAAV-based gene therapy to be approved in the Western world (Glybera® for lipoprotein lipase deficiency, approved for use in 2012 in the European Union) has stimulated the gene therapy community, investors and regulators to the real possibility of moving rAAV therapies into the clinic globally. Yet, despite the impressive abilities of rAAV to transduce a variety of tissue and cell types, skeletal muscle has been historically been one of the most challenging tissues to transduce at high levels sufficient to provide therapeutic levels of expression of delivered transgene products.

A variety of published US applications describe AAV vectors and virions, including U.S. Pat. Nos. 9,587,250; 9,457,103; and 8,663,324; and U.S. Publication Nos. 2017/0159026; 2015/0023924; and 2014/0348794, all of which are incorporated by reference herein in their entireties.

There remains, therefore, a need in the art for AAV vectors with improved human liver and pancreas transduction. The present invention meets this need by providing variant AAV capsid polypeptides which demonstrate significantly improved human liver and pancreas transduction over existing capsid serotypes. The present invention utilizes designed ankyrin repeat proteins (DARPins) to engineer novel variant capsid polypeptides that have high transduction efficiency and tissue tropism for human liver or pancreas.

### BRIEF SUMMARY OF THE INVENTION

The present invention provides a variant adeno-associated virus (AAV) capsid polypeptide comprising a designed ankyrin repeat protein (DARPin) or fragment thereof fused to the N-terminus of an AAV capsid protein VP2, wherein said DARPin specifically binds to a cell surface molecule expressed on human liver tissue or cells and said variant capsid polypeptide exhibits increased transduction or tropism in human liver tissue or cells as compared to a non-variant parent capsid polypeptide, or wherein said DARPin specifically binds to a cell surface molecule expressed on human pancreatic tissue or cells and said variant capsid polypeptide exhibits increased transduction or tropism in human pancreatic tissue or cells as compared to a non-variant parent capsid polypeptide.

In some embodiments, the variant capsid polypeptide further exhibits an enhanced neutralization profile as compared to a non-variant parent capsid polypeptide.

In some embodiments, the variant capsid polypeptide said variant capsid polypeptide further exhibits increased transduction or tropism in one or more non-liver human tissues or one or more non-pancreatic human tissues as compared to a non-variant parent capsid polypeptide.

In some embodiments, the cell surface molecule expressed on human liver tissue or cells is asialoglycoprotein receptor (ASGPR) or said cell surface molecule expressed on human pancreatic tissue or cells is CD200. In some embodiments, the variant capsid polypeptide comprises an amino acid sequence having at least 85% sequence identity to the sequence selected from the group consisting of SEQ ID NOs: 1 to 4.

Any one of the variant capsid polypeptides described herein can be part of a functional AAV capsid. In certain embodiments, the functional AAV capsid packages a nucleic acid sequence selected from the group consisting of a non-coding RNA, a protein coding sequence, an expression cassette, a multi-expression cassette, a sequence for homologous recombination, a genomic gene targeting cassette, and a therapeutic expression cassette. The nucleic acid sequence can be contained within an AAV vector. Optionally, expression cassette is a CRISPR/CAS expression system. In some instances, the therapeutic expression cassette encodes a therapeutic protein or antibody.

Also provided are methods of using any variant AAV capsid polypeptide outlined herein in a therapeutic treatment regimen or vaccine. In some embodiments, the method can reduce the amount of total nucleic acid administered to a subject. The method can include administering less total nucleic acid amount to the subject when the nucleic acid is transduced using a variant capsid polypeptide as compared to the amount of nucleic acid administered to the subject when said nucleic acid is transduced using a non-variant parent capsid polypeptide in order to obtain a similar therapeutic effect.

The present invention provides an adeno-associated virus (AAV) vector comprising a nucleic acid sequence encoding a variant capsid polypeptide comprising a designed ankyrin repeat protein (DARPin) or fragment thereof fused to the N-terminus of an AAV capsid protein VP2, wherein said DARPin specifically binds to a cell surface molecule expressed on human liver tissue or cells and said variant capsid polypeptide exhibits increased transduction or tropism in human liver tissue or cells as compared to a non-variant parent capsid polypeptide, or wherein said DARPin specifically binds to a cell surface molecule expressed on human pancreatic tissue or cells and said variant capsid polypeptide exhibits increased transduction or tropism in human pancreatic tissue or cells as compared to a non-variant parent capsid polypeptide.

In some embodiments, the variant capsid polypeptide exhibits increased transduction as compared to a non-variant parent capsid polypeptide. In certain embodiments, the variant capsid polypeptide further exhibits an enhanced neutralization profile as compared to a non-variant parent capsid polypeptide.

In other embodiments, the variant capsid polypeptide further exhibits increased transduction or tropism in one or more non-liver human tissues or one or more non-pancreatic human tissues as compared to a non-variant parent capsid polypeptide.

In some embodiments, the cell surface molecule expressed on human liver tissue or cells is asialoglycoprotein receptor (ASGPR), or said cell surface molecule expressed on human pancreatic tissue or cells is CD200. In some instances, the variant capsid polypeptide comprises an amino acid sequence having at least 85% sequence identity to the sequence selected from the group consisting of SEQ ID NOs: 1 to 4.

The present invention also provides an adeno-associated virus (AAV) vector containing a nucleic acid sequence encoding a variant capsid polypeptide comprising a designed ankyrin repeat protein (DARPin) or fragment thereof fused to the N-terminus of an AAV capsid protein VP2. In some instances, the DARPin specifically binds to a cell surface molecule expressed on human pancreatic tissue or cells. In some cases, the cell surface molecule expressed on the human pancreatic tissue or cells is CD200. In certain embodiments, the variant capsid polypeptide comprises an amino acid sequence selected from the group consisting of SEQ ID NOs:1 to 4. In some embodiments, variant capsid polypeptide exhibits increased transduction or tropism in human pancreatic tissue or cells as compared to a non-variant parent capsid polypeptide.

In some embodiments, the AAV vector further includes a nucleic acid sequence selected from the group consisting of a non-coding RNA, a coding sequence, an expression cassette, a multi-expression cassette, a sequence for homologous recombination, a genomic gene targeting cassette, and a therapeutic expression cassette. The variant capsid polypeptide allows for nucleic acid expression similarly to a non-variant parent capsid polypeptide. In some instances, the expression cassette is a CRISPR/CAS expression system. In some instances, the therapeutic expression cassette encodes a therapeutic protein or antibody.

In one aspect, the present invention describes a method of using any of the AAV vectors disclosed herein in a therapeutic treatment regimen or vaccine. In another aspect, a method of using any of the AAV vectors to reduce the amount of total AAV vector administered to a subject is provided herein. The method can include administering less total AAV vector amount to the subject when the AAV vector is transduced by a variant capsid polypeptide, as compared to the amount of AAV vector administered to the subject when the AAV vector is transduced by a non-variant parent capsid polypeptide in order to obtain a similar therapeutic effect.

In some aspects, the present invention provides methods for generating a variant AAV capsid polypeptide comprising a designed ankyrin repeat protein (DARPin) or fragment thereof fused to the N-terminus of an AAV capsid protein VP2, wherein the DARPin specifically binds to a cell surface molecule expressed on human liver or pancreatic tissue or cells, wherein the variant capsid polypeptide exhibits increased transduction or tropism in human liver or pancreatic tissue or cells as compared to a non-variant parent capsid polypeptide. The method includes: (a) generating a library of variant capsid polypeptide genes, wherein said variant capsid polypeptide genes include a plurality of variant capsid polypeptide genes comprising sequences from more than one non-variant parent capsid polypeptide; (b) generating an AAV vector library by cloning said variant capsid polypeptide gene library into AAV vectors, wherein said AAV vectors are replication competent AAV vectors; (c) screening said AAV vector library from b) for variant AAV capsid polypeptides for increased transduction or tropism in human liver and pancreatic tissue or cells as compared to a non-variant parent capsid polypeptide; and (d) selecting said variant AAV capsid polypeptides from (c).

In some embodiments, the method also includes performing (c) and (d) one or more times. In certain embodiments, the method further includes (e) determining the sequence of said variant capsid polypeptides from (d). In some embodiments, the method also includes transducing target cells with the AAV vector library. The target cells can be recombinant cells expressing a cell surface molecule expressed on human liver or pancreatic tissue or cells. In some embodiments, the cell surface molecule expressed on human liver tissue or cells is ASGPR or the cell surface molecule expressed on human pancreatic tissue or cells is CD200.

In some embodiments, the variant capsid polypeptide exhibits increased transduction as compared to a non-variant parent capsid polypeptide. In some embodiments, the variant capsid polypeptide exhibits increased tropism as compared to a non-variant parent capsid polypeptide. In some embodiments, the variant capsid polypeptide exhibits increased tropism as compared to a non-variant parent capsid polypeptide. In some embodiments, the variant capsid polypeptide further exhibits increased transduction or tropism in one or more non-liver or non-pancreatic human tissues as compared to a non-variant parent capsid polypeptide. In some embodiments, the variant capsid polypeptide exhibits increased transduction of human liver or pancreatic tissue or cells in vivo as compared to a non-variant parent capsid polypeptide. In some embodiments, the variant capsid polypeptide exhibits increased transduction of human liver or pancreatic tissue or cells in vitro as compared to a non-variant parent capsid polypeptide. In some embodiments, the variant capsid polypeptide exhibits increased transduction of human liver or pancreatic tissue or cells ex vivo as compared to a non-variant parent capsid polypeptide.

Other objects, advantages and embodiments of the invention will be apparent from the detailed description following.

### BRIEF DESCRIPTION OF THE DRAWINGS

FIG. 1A and FIG. 1B illustration construction of the DARPin-AAV library system. FIG. 1A shows open reading frames of AAV-2 and AAV-DJ cap were mutated for the VP2 start codon. FIG. 1B shows that DARPin N3C libraries were fused in frame with respective VP2 reading frames containing VP2 start site mutations as indicated in FIG. 1A.

FIG. 2A and FIG. 2B provide library cell lines. CHO-k1 cells were transfected with expression plasmids containing either the extracellular domain of ASGPR-1 (murine) or CD200 (human) along with a neomycin selection cassette. Cells were subsequently selected with G418 and FACS sorted for receptor positive cells. FIG. 2A shows ASGPR-expressing cells detected with a myc-tag specific antibody. FIG. 2B shows CD200 expressing cells detected with an antibody directly recognizing the receptor.

FIG. 3 depicts a schematic of library selection. AAV-DARPin-lib particles were produced by transient transfection of 293T cells with the library plasmid cloned into an AAV vector genome (containing ITR sequences), the packaging plasmid and an adenoviral helper plasmid. Receptor negative CHO-k1 cells were incubated with purified AAV-DARPin-lib stocks for 6-12 hours (pre-screening) and supernatant was subsequently transferred to respective CHO cells expressing the desired receptor. After 24 hours cell were extensively washed and genomic DNA was isolated, sub-cloned into the library plasmid and a new selection cycle was initiated.

FIG. 4A and FIG. 4B shows enrichment of AAV-DARPin pools. After each selection round individual DARPin pools were rescued with rescue primers and sub-cloned into the library plasmid (FIG. 4A). Individually selected DARPin pools show increasing signal intensity after each selection round (red arrow), indicating successful enrichment of selected DARPin pools (FIG. 4B).

FIG. 5A and FIG. 5B show analysis of individual selection rounds. FIG. 5A shows selected DARPin sequences. After each selection round and sub-cloning into the library plasmid, 100 clones from each pool were sequenced and aligned. Individual repeat motifs were analyzed for recurring sequence motifs. FIG. 5B shows that the number of identical repeat motifs increased from selection round 1-5, indicating successful selection of specific DARPins.

FIG. 6 depicts exemplary identified DARPin molecules that bind CD200. After 5 selection rounds, the CD200 selection pool revealed two DARPin molecules (DARPin-CD200-19, DARPin-CD200-23). The DARPins had identical amino acid sequences, except at 7 residues in the C-terminal repeat motif (repeat 3) and 2 residues in the C-cap. The amino acid sequences of the exemplary DARPins of FIG. 6 are set forth in SEQ ID NO: 11-13.

FIG. 7 illustrates transduction of AAV-DARPin-CD200-19 and AAV-DARPin-CD200-23 into receptor positive cells (CHO-CD200 target cells), and not non-target cells (CHO-k1 cells).

FIG. 8A and FIG. 8B show construction of the replicating DARPin-AAV system. The helper AAV was constructed by mutating the AAV-2 VP2 start site, thereby creating a self-replicating virus lacking the VP2 protein (FIG. 8A). The replicating DARPin-AAV was generated by fusing the DARPin sequence in frame to the VP2 reading frame, thus deleting partial sequences of the VP1 reading frame. FIG. 8B shows a detailed illustration of the rep-DARPin intergenic sequence (boxed region in FIG. 8A). The rep intron was conserved during the cloning process to allow expression of rep40 and rep68.

FIG. 9A and FIG. 9B illustrate replication of the self-replicating DARPin-AAV. FIG. 9A provides a schematic of primers used for DARPin sequence detection. SK-OV-3 cells were transduced with self-replicating DARPin-AAV and infected with adenovirus (Ad) 6 hours after transduction. After 3 days cells were harvested and genomic DNA was isolated. Replication was detected using primers indicated in FIG. 9A. FIG. 9B shows integration of the replicating DARPin-AAV vector when the cells were infected with adenovirus (Ad).

FIG. 10 depicts the use of adeno-associated viral vectors for gene therapy.

FIG. 11A-FIG. 11D provide exemplary embodiments of DARPins. See, e.g., Binz et al., J Mol Biol, 2003, 332(2):489-503 and Amstutz et al., J Biol Chem, 2005, 280-(26):24715-22.

FIG. 12 shows AAV-2 vectors modified to contain DARPins. Exemplary DARPins can provide a novel targeting domain for AAV-2. See, e.g., Munch et al., Nat Commun, 2015, 6:6246. DARPin targeting can be applied to shuffled AAV capsids that show much higher transduction efficiencies compared to wild-type capsids. Thus, DARPins can be used to target different cells, including difficult to transduce cell types.

FIG. 13 provides a schematic diagram of a DARPin selection system using ribosomal display. This system is based on artificial DARPin-target interactions. Ribosomal display requires highly purified target proteins and large pools after multiple selection rounds. Few DARPins are suitable for AAV incorporation.

FIG. 14 shows the novel DARPin selection method described herein.

FIG. 15 shows that DARPin targeting is superior on shuffled AAV capsids.

FIG. 16 provides a schematic diagram of an embodiment of the DARPin selection method of the present invention. DARPin AAV libraries can be produced with adequate diversity and at desired titers. Certain repeat motifs responsible for target receptor binding can be significantly enrich in a few selection rounds. This selection strategy is advantageous in terms of time, cost and efficiency.

FIG. 17A and FIG. 17B show DARPin targets such as CD200 (FIG. 17A) and asialoglycoprotein receptor (ASGPR; FIG. 17B).

### DETAILED DESCRIPTION OF THE INVENTION

### Introduction

The present invention is based, in part, on the incorporation of highly diverse DARPin libraries into AAV capsids using a novel VP2-fusion protein. Replication-deficient and replication-competent AAV libraries were generated with high diversity, functional titers, and specific infectivity of therapeutically relevant cell types. Provided herein are DARPin-AAV-DJ capsid and DARPin-AAV-2 capsid libraries that underwent multiple rounds of selection to enrich AAV capsids with high binding affinity to specific cell types, such as CD200 positive pancreatic islet cells and ASGPR positive liver cells. Also, provided herein are DARPin-capsids that selectively bind to CD200, a membrane glycoprotein that is specifically expressed on pancreatic islet cells. DARPin-capsids that selectively bind to the asialoglycoprotein receptor (ASGPR) which is specifically expressed on liver cells (e.g., hepatocytes). The method can expand the potential diversity of AAV vectors by creating novel DARPin-AAV vectors with medically relevant transduction properties.

### Abbreviations

“AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”).

### Definitions

The term “DARPin” refers to designed ankyrin repeat proteins based on naturally occurring ankyrin repeat proteins. DARPins are selected for high-affinity binding to a target molecule. An ankyrin repeat module includes about 33 amino acid residues and by combining 2-3 of these repeats and flanking them by N- and C-capping repeats.

The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV 9_hu14, avian AAV, bovine AAV, canine AAV, equine AAV, primate AAV, non-primate AAV, ovine AAV, and AAV-DJ. “AAV-DJ” refers to a AAV type 2/type 8/type 9 chimera that is distinguished from it closest natural relative AAV-2 by 60 capsid amino acids (see, e.g., Grimm et al., J. Virology, 2008, 82(12):5887-5911). “Primate AAV” refers to AAV capable of infecting primates, “non-primate AAV” refers to AAV capable of infecting non-primate mammals, “bovine AAV” refers to AAV capable of infecting bovine mammals, etc.

An “AAV vector” as used herein refers to an AAV vector nucleic acid sequence encoding for various nucleic acid sequences, including in some embodiments a variant capsid polypeptide (i.e., the AAV vector comprises a nucleic acid sequence encoding for a variant capsid polypeptide), wherein the variant capsid polypeptide exhibits increased transduction or tropism in human liver tissue or cells as compared to a non-variant parent capsid polypeptide. The AAV vectors can also comprise a heterologous nucleic acid sequence not of AAV origin as part of the nucleic acid insert. This heterologous nucleic acid sequence typically comprises a sequence of interest for the genetic transformation of a cell. In general, the heterologous nucleic acid sequence is flanked by at least one, and generally by two AAV inverted terminal repeat sequences (ITRs).

The phrase “non-variant parent capsid polypeptide” includes any naturally occurring AAV capsid polypeptide and/or any AAV wild-type capsid polypeptide. In some embodiments, the non-variant parent capsid polypeptide includes AAV1, AAV2, AAV3, AAV4, AAV5, AAV6, AAV7, AAV8, AAV9, bovine AAV and/or avian AAV capsid polypeptides.

The term “substantially identical” in the context of variant capsid polypeptides and non-variant parent capsid polypeptides refers to sequences with 1 or more amino acid changes. In some embodiments, these changes do not affect the packaging function of the capsid polypeptide. In some embodiments, substantially identical include variant capsid polypeptides about 99%, about 98%, about 97%, about 96%, about 95%, about 94%, about 93%, about 92%, about 91%, or about 90% identical to a non-variant parent capsid polypeptide. In some embodiments, the variant capsid polypeptide can be substantially identical to a non-variant parent capsid polypeptide over a subregion of the variant capsid polypeptide, such as over about 25%, about 50%, about 75%, or about 90% of the total polypeptide sequence length.

An “AAV virion” or “AAV virus” or “AAV viral particle” or “AAV vector particle” refers to a viral particle composed of at least one AAV capsid polypeptide (including both variant capsid polypeptides and non-variant parent capsid polypeptides) and an encapsidated polynucleotide AAV transfer vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle.

“Packaging” refers to a series of intracellular events resulting in the assembly of AAV virions or AAV particles which encapsidate a nucleic acid sequence and/or other therapeutic molecule. Packaging can refer to encapsidation of nucleic acid sequence and/or other therapeutic molecules into a capsid comprising the variant capsid polypeptides described herein.

The phrase “therapeutic molecule” as used herein can include nucleic acids (including, for example, vectors), polypeptides (including, for example, antibodies), and vaccines, as well as any other therapeutic molecule that could be packaged by the variant AAV capsid polypeptides of the invention.

AAV “rep” and “cap” genes refer to polynucleotide sequences encoding replication and encapsidation proteins of adeno-associated virus (AAV). AAV rep (replication) and cap (capsid) are referred to herein as AAV “packaging genes.”

A “helper virus” for AAV refers to a virus allowing AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used as a helper virus. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome allowing AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g. a replication-competent AAV) refers to an infectious phenotypically wild-type virus, and is replicable in an infected cell (i.e. in the presence of a helper virus or helper virus functions). In the case of AAV, replication competence generally requires the presence of functional AAV packaging genes. In some embodiments, AAV vectors, as described herein, lack of one or more AAV packaging genes and are replication-incompetent in mammalian cells (especially in human cells). In some embodiments, AAV vectors lack any AAV packaging gene sequences, minimizing the possibility of generating replication competent AAV by recombination between AAV packaging genes and an incoming AAV vector. In many embodiments, AAV vector preparations as described herein are those containing few if any replication competent AAV (rcAAV, also referred to as RCA) (e.g., less than about 1 rcAAV per 10² AAV particles, less than about 1 rcAAV per 10⁴ AAV particles, less than about 1 rcAAV per 10⁸ AAV particles, less than about 1 rcAAV per 10¹² AAV particles, or no rcAAV).

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “gene” refers to a polynucleotide containing at least one open reading frame capable of encoding a particular protein or polypeptide after being transcribed and translated.

“Recombinant,” as applied to a polynucleotide means the polynucleotide is the product of various combinations of cloning, restriction or ligation steps, and other procedures resulting in a construct distinct and/or different from a polynucleotide found in nature. A recombinant virus is a viral particle encapsidating a recombinant polynucleotide. The terms respectively include replicates of the original polynucleotide construct and progeny of the original virus construct.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

“Heterologous” means derived from a genotypically distinct entity from the rest of the entity to it is being compared too. For example, a polynucleotide introduced by genetic engineering techniques into a plasmid or vector derived from a different species is a heterologous polynucleotide. A promoter removed from its native coding sequence and operatively linked to a coding sequence it is not naturally found linked to a heterologous promoter. For example, an AAV including a heterologous nucleic acid encoding a heterologous gene product is an AAV including a nucleic acid not normally included in a naturally-occurring, wild-type AAV, and the encoded heterologous gene product is a gene product not normally encoded by a naturally-occurring, wild-type AAV. An AAV including a nucleic acid encoding a variant capsid polypeptide includes a heterologous nucleic acid sequence. Once transferred/delivered into a host cell, a heterologous polynucleotide, contained within the virion, can be expressed (e.g., transcribed, and translated if appropriate). Alternatively, a transferred/delivered heterologous polynucleotide into a host cell, contained within the virion, need not be expressed. Although the term “heterologous” is not always used herein in reference to polynucleotides, reference to a polynucleotide even in the absence of the modifier “heterologous” is intended to include heterologous polynucleotides in spite of the omission.

The terms “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell; but any alteration changing the phenotype and/or genotype of the cell and its progeny is included in this term.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced and inheritable by progeny of the altered cell.

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. In methods and uses of as described herein, such polypeptides, proteins and peptides encoded by the polynucleotide sequences can be but are not required to be identical to the defective endogenous protein, or whose expression is insufficient, or deficient in the treated mammal. The terms also encompass a modified amino acid polymer; for example, disulfide bond formation, glycosylation, lipidation, phosphorylation, methylation, carboxylation, deamidation, acetylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, retaining the desired biochemical function of the intact protein.

As used herein, the abbreviations for the genetically encoded L-enantiomeric amino acids used in the disclosure methods are conventional and are as follows.

“Hydrophilic Amino Acid” refers to an amino acid exhibiting a hydrophobicity of less than zero according to the normalized consensus hydrophobicity scale of Eisenberg et al., 1984, J. Mol. Biol. 179: 125-142. Genetically encoded hydrophilic amino acids include Thr (T), Ser (S), His (H), Glu (E), Asn (N), Gln (Q), Asp (D), Lys (K) and Arg I.

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic Amino Acid” refers to a hydrophilic amino acid having a side chain pK value of greater than 7. Basic amino acids typically have positively charged side chains at physiological pH due to association with hydrogen ion. Genetically encoded basic amino acids include His (H), Arg (R) and Lys (K).

“Polar Amino Acid” refers to a hydrophilic amino acid having a side chain uncharged at physiological pH, but which has at least one bond in which the pair of electrons shared in common by two atoms is held more closely by one of the atoms. Genetically encoded polar amino acids include Asn (N), Gln (Q), Ser (S) and Thr (T).

“Hydrophobic Amino Acid” refers to an amino acid exhibiting a hydrophobicity of greater than zero according to the normalized consensus hydrophobicity scale of Eisenberg, 1984, J. Mol. Biol. 179:125-142. Exemplary hydrophobic amino acids include Ile (I), Phe (F), Val (V), Leu (L), Trp (W), Met (M), Ala (A), Gly (G), Tyr (Y), Pro (P), and proline analogues.

“Aromatic Amino Acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO2, —NO, —NH₂, —NHR, —NRR, —C(O)R, —C(O)OH, —C(O)OR, —C(O)NH2, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₁-C₂₁)) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

“Nonpolar Amino Acid” refers to a hydrophobic amino acid having a side chain uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic Amino Acid” refers to a hydrophobic amino acid having an aliphatic hydrocarbon side chain. Genetically encoded aliphatic amino acids include Ala (A), Val (V), Leu (L) and Ile (I).

The term “non-naturally” with regard to amino acids can include any amino acid molecule not included as one of the 20 amino acids listed in Table 1 above as well as any modified or derivatized amino acid known to one of skill in the art. Non-naturally amino acids can include but are not limited to β-alanine, α-amino butyric acid, γ-amino butyric acid, γ-(aminophenyl) butyric acid, α-amino isobutyric acid, ε-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

The term “variant” or “variants”, with regard to polypeptides, such as capsid polypeptides refers to a polypeptide sequence differing by at least one amino acid from a parent polypeptide sequence, also referred to as a non-variant polypeptide sequence. In some embodiments, the polypeptide is a capsid polypeptide and the variant differs by at least one amino acid substitution. Amino acids also include naturally occurring and non-naturally occurring amino acids as well as derivatives thereof. Amino acids also include both D and L forms.

An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

By the term “highly conserved” is meant at least about 80% identity, preferably at least 90% identity, and more preferably, over about 97% identity. Identity is readily determined by one of skill in the art by resort to algorithms and computer programs known by those of skill in the art.

The term “percent sequence identity” or “identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to about 24 nucleotides, at least about 28 to about 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, and may be up to about 700 amino acids.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an AAV vector or AAV virion as disclosed herein, or transformed cell to a subject.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. AAV vectors or AAV virions, and pharmaceutical compositions thereof can be packaged in single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue of a subject (for example, liver tissue), a therapeutically effective dose will be on the order of from about 10⁶ to about 10¹⁵ of the AAV virions, e.g., from about 10⁸ to 10¹² AAV virions. Other effective dosages can be readily established by one of ordinary skill in the art through routine trials establishing dose response curves.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

The phrase “enhanced neutralization profile” refers to the ability of an AAV vector or virion to better evade neutralizing antibody binding in the subject. In some instances, fewer neutralization antibodies allow for the AAV infection to allow for higher levels of transduction, making the variant AAV capsid polypeptides, AAV vectors and virions of the present invention better suited for gene therapy purposes.

The phrases “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate.

The term “tropism profile” refers to the pattern of transduction of one or more target cells, tissues and/or organs. For example, some shuffled AAV capsids (variant AAV capsid polypeptides) provide for efficient transduction of liver tissue or liver cells such as, but not limited to, islet cells. Conversely, some shuffled AAV capsids have only low level transduction of liver, gonads and/or germ cells. The variant AAV capsid polypeptides disclosed herein provide for efficient and/or enhanced transduction of liver and islet cells including CD200 positive islet cells.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 110%, 125%, 150%, 175%, or 200% or more of the transduction or tropism, respectively, of the control). Suitable controls will depend on a variety of factors including the desired tropism profile. Similarly, it can be determined if a capsid and/or virus “does not efficiently transduce” or “does not have efficient tropism” for a target tissue, or similar terms, by reference to a suitable control.

As used herein and in the appended claims, the singular forms “a,” “an,” and “the” include plural referents unless the context clearly dictates otherwise. Thus, for example, reference to “an AAV virion” includes a plurality of such virions and reference to “a host cell” includes reference to one or more host cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Before the invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### AAV Capsid and Vector Features

AAV vectors of the present invention on have numerous features. In some embodiments, the vectors comprise nucleic acid sequences encoding for variant capsid polypeptides. Such AAV vectors and their features are described in detail below.

An exemplary AAV vector of the present invention comprise a nucleic acid encoding for a variant AAV capsid protein differing in amino acid sequence by at least one amino acid from a wild-type or non-variant parent capsid protein. The amino acid difference(s) can be located in a solvent accessible site in the capsid, e.g., a solvent-accessible loop, or in the lumen (i.e., the interior space of the AAV capsid). In some embodiments, the lumen includes the interior space of the AAV capsid. For example, the amino acid substitution(s) can be located in a GH loop in the AAV capsid polypeptide. In some embodiments, the variant capsid polypeptide comprises an amino acid substitution in AAV-DJ, AAV1, AAV2, AAV3, AAV4, AAV5, AAV6, AAV7, AAV8, or AAV9 capsid polypeptides.

In some embodiments, the present invention provides an isolated nucleic acid comprising a nucleotide sequence that encodes a variant adeno-associated virus (AAV) capsid protein, where the variant AAV capsid protein comprises an amino acid sequence having at least about 85% at least about 90%, at least about 95%, at least about 98%, at least about 99%, or 100%, to non-variant capsid amino acid sequences or to sub-portions of a non-variant parent capsid polypeptide sequence, and exhibits increased transduction or tropism in human liver or pancreatic tissue or cells as compared to a vector encoding a non-variant parent capsid polypeptide. In some embodiments, the present invention provides an isolated nucleic acid comprising a nucleotide sequence that encodes a variant adeno-associated virus (AAV) capsid protein, where the variant AAV capsid protein comprises an amino acid sequence having at least about 85% at least about 90%, at least about 95%, at least about 98%, at least about 99%, or 100%, to parental non-variant capsid amino acid sequences or to sub-portions of a non-variant parent capsid polypeptide sequence, such as wild-type AAV-DJ, AAV1, AAV2, AAV3, AAV4, AAV5, AAV6, AAV7, AAV8, or AAV9 capsid polypeptides, and where the variant capsid polypeptide exhibits increased transduction or tropism in human liver or pancreatic tissue or cells as compared to a vector encoding a non-variant parent capsid polypeptide. In some embodiments, the variant capsid polypeptide comprises one or more regions or sub-portions from non-variant parent capsid polypeptide sequences from AAV serotypes 1, 2, 6, 8, 9, and chimeric variants thereof (i.e., AAV1, AAV2, AAV6, AAV8, AAV9, and AAV-DJ).

In some embodiments, a subject AAV vector can encode a variant capsid polypeptide having an amino acid sequence of at least about 85%, at least about 90%, at least about 95%, at least about 98%, or at least about 99%, or 100%, amino acid sequence identity to non-variant parent capsid polypeptide or to sub-portions of a non-variant parent capsid polypeptide. In some embodiments, the variant capsid polypeptide is encoded by other vectors/plasmids known in the art.

In some embodiments, the variant AAV capsid polypeptide sequence comprises any one of SEQ ID NOS: 5, 6, and 9. In some embodiments, the variant AAV capsid polypeptide sequence is encoded by any one of SEQ ID NOS: 7, 8 and 10.

In some embodiments, the variant capsid polypeptides exhibit substantial homology or “substantial similarity,” when referring to amino acids or fragments thereof, indicates that, when optimally aligned with appropriate amino acid insertions or deletions with another amino acid (or its complementary strand), there is amino acid sequence identity in at least about 95% to about 99% of the aligned sequences. In some embodiments, the homology is over full-length sequence, or a polypeptide thereof, e.g., a capsid protein, or a fragment thereof of at least 8 amino acids, or more desirably, at least about 15 amino acids in length, including sub-portions of a non-variant parent capsid polypeptide sequence. For example, the variant capsid polypeptide can comprise an amino acid sequence having at least about 85%, at least about 90%, at least about 95%, at least about 98%, at least about 99%, or 100%, amino acid sequence identity to a non-variant parent capsid polypeptide sequence or to sub-portions of a non-variant parent capsid polypeptides. In some embodiments, the variant capsid polypeptide sequence comprises SEQ ID NOS:5 or 6. In some embodiments, the variant capsid polypeptide sequence is encoded by SEQ ID NOS:7 or 8.

In some embodiments, the variant AAV capsid polypeptides of the invention exhibit increased transduction in human liver tissue or cells as compared to a non-variant parent capsid polypeptide. In some embodiments, the variant AAV capsid polypeptides of the invention exhibit increased transduction in human pancreatic tissue or cells as compared to a non-variant parent capsid polypeptide. In some embodiments, transduction is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction is increased by about 5% to about 80%, about 10% to about 70%, about 20% to about 60%, about 30% to about 60%, or about 40% to about 50%.

In some embodiments, the variant AAV capsid polypeptides of the invention exhibit increased tropism in human liver tissue or cells as compared to a non-variant parent capsid. In some embodiments, the variant AAV capsid polypeptides of the invention exhibit increased tropism in human pancreatic tissue or cells as compared to a non-variant parent capsid. In some embodiments, tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, tropism is increased by about 5% to about 80%, about 10% to about 70%, about 20% to about 60%, about 30% to about 60%, or about 40% to about 50%.

In some embodiments, the variant AAV capsid polypeptides of the invention further exhibit an enhanced neutralization profile as compared to a non-variant parent capsid polypeptide. In some embodiments, the variant capsid polypeptide of the invention further exhibits an enhanced neutralization profile against pooled human immunoglobulins as compared to a non-variant parent capsid polypeptide. In some embodiments, the neutralization profile is enhanced by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, the neutralization profile is enhanced by about 5% to about 80%, about 10% to about 70%, about 20% to about 60%, about 30% to about 60%, or about 40% to about 50%. In some embodiments, an enhanced neutralization profile is determined by a reduction in the generation of neutralizing antibodies in a host. In some embodiments, the reduction in generation of neutralizing antibodies is a reduction of about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, the reduction in generation of neutralizing antibodies is a reduction of about 5% to about 80%, about 10% to about 70%, about 20% to about 60%, about 30% to about 60%, or about 40% to about 50%.

In some embodiments, the variant AAV capsid polypeptides of the invention further exhibit increased transduction or tropism in one or more human stem cell types as compared to a non-variant parent capsid polypeptide. In some embodiments, the human stem cell types include but are not limited to embryonic stem cells, adult tissue stem cells (i.e., somatic stem cells), bone marrow, progenitor cells, induced pluripotent stem cells, and reprogrammed stem cells. In some embodiments, adult stem cells can include organoid stem cells (i.e., stem cells derived from any organ or organ system of interest within the body). Organs of the body include for example but are not limited to skin, hair, nails, sense receptors, sweat gland, oil glands, bones, muscles, brain, spinal cord, nerve, pituitary gland, pineal gland, hypothalamus, thyroid gland, parathyroid, thymus, adrenals, pancreas (islet tissue), heart, blood vessels, lymph nodes, lymph vessels, thymus, spleen, tonsils, nose, pharynx, larynx, trachea, bronchi, lungs, mouth, pharynx, esophagus, stomach, small intestine, large intestine, rectum, anal canal, teeth, salivary glands, tongue, liver, gallbladder, pancreas, appendix, kidneys, ureters, urinary bladder, urethra, testes, ductus (vas) deferens, urethra, prostate, penis, scrotum, ovaries, uterus, uterine (fallopian) tubes, vagina, vulva, and mammary glands (breasts). Organ systems of the body include but are not limited to the integumentary system, skeletal system, muscular system, nervous system, endocrine system, cardiovascular system, lymphatic system, respiratory system, digestive system, urinary system, and reproductive system. In some embodiments, transduction and/or tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction and/or tropism is increased by about 5% to about 80%, about 10% to about 70%, about 20% to about 60% or about 30% to about 60%.

In some embodiments, the variant AAV capsid polypeptides of the invention further exhibit increased transduction or tropism in one or more non-liver human tissues as compared to a non-variant parent capsid polypeptide. In some embodiments, the variant capsid polypeptide further exhibits increased transduction in one or more non-liver human tissues as compared to a vector encoding a non-variant parent capsid polypeptide. In some embodiments, the variant capsid polypeptide further exhibits increased tropism in one or more non-liver human tissues as compared to a vector encoding a non-variant parent capsid polypeptide. In some embodiments, transduction and/or tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction and/or tropism is increased by about 5% to about 80%, about 10% to about 70%, about 20% to about 60% or about 30% to about 60%.

In some embodiments, the variant AAV capsid polypeptides of the invention further exhibit increased transduction or tropism in one or more non-liver human tissues as compared to a non-variant parent capsid polypeptide. In some embodiments, the variant capsid polypeptide further exhibits increased transduction in one or more non-liver human tissues as compared to a vector encoding a non-variant parent capsid polypeptide. In some embodiments, the variant capsid polypeptide further exhibits increased tropism in one or more non-liver human tissues as compared to a vector encoding a non-variant parent capsid polypeptide. In some embodiments, transduction and/or tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction and/or tropism is increased by about 5% to about 80%, about 10% to about 70%, about 20% to about 60% or about 30% to about 60%.

In some embodiments, the variant capsid polypeptide contains a VP1 comprising AAV2. In other embodiments, the variant capsid polypeptide contains a VP1 comprising AAV-DJ. In some embodiments, the variant capsid polypeptide contains a VP2 comprising AAV2. In other embodiments, the variant capsid polypeptide contains a VP2 comprising AAV-DJ. In some embodiments, the variant capsid polypeptide contains a VP3 comprising AAV2. In some embodiments, the variant capsid polypeptide contains a VP3 comprising AAV-DJ. In other embodiments, the variant capsid polypeptide contains a VP3 comprising AAV1, AAV2, AAV8, and/or AAV-DJ. In some embodiments, the variant capsid polypeptide contains a VP1 comprising AAV-DJ, AAV2, AAV3b and AAV8. In some embodiments, the variant capsid polypeptide contains a VP2 comprising AAV-DJ, AAV2, AAV3b, and/or AAV9. In some embodiments, the variant capsid polypeptide contains a VP3 comprising AAV-DJ, AAV2, AAV3b, and/or AAV8.

In some embodiments, the variant capsid polypeptide sequence comprises a sequence selected from the group consisting of AAV-NP66 (SEQ ID NO:5), AAV-NP22 (SEQ ID NO:6), and AAV-NP94 (SEQ ID NO:9). For NP66, the unique region of VP1 is composed of AAV1, VP2 is composed of AAV1, and VP3 is composed of AAV1, AAV2, and AAV8. For NP22, the unique region of VP1 is composed of AAV3b and AAV8, VP2 is composed of AAV2, AAV3b, and AAV9, VP3 is composed of AAV2, AAV3b, and AAV8.

### Designed Ankyrin Repeat Proteins (DARPins)

Designed ankyrin repeat proteins (DARpins) can be included in synthetic, variant AAV capsid proteins to improve transduction efficiency and/or tropism in specific human tissues, e.g., liver tissue or pancreatic tissue. One or more synthetic DARPins can be fused to viral proteins on the virion shell. DARPins are based on naturally occurring ankyrin repeat proteins, yet contain one or more amino acid mutations that can affect, for example, its binding affinity to a target molecule, its cell surface expression, and the like. DARPins can include 2 to 3 ankyrin repeat modules flanked by N- and C-capping repeats, which can be referred to as N2C or N3C. Each ankyrin repeat module contains about 33 amino acid residues that interact the target molecule, e.g., target protein or target receptor.

DARPins that bind to specific targets can be identified by screening combinatorial libraries of DARPins and selecting those with desired binding properties for the target. Such screening methods are described in, e.g., Muench et al., Molecular Therapy, 16(4), 686-693, 2011. For example, ribosomal display or phage display methods can be used to select target-specific DARPins from diverse libraries.

Provided herein are DARPins fused to AAV capsid proteins that specifically bind to CD200 or to ASGPR. The DARPins were identified after several rounds of screening of diverse DARPin libraries.

### Generating AAV Capsid Polypeptides

The present invention also provides methods for generating engineered, variant capsid polypeptides. In some cases, the capsid polypeptides are based on AAV-DJ, AAV1, AAV2, AAV3, AAV4, AAV5, AAV6, AAV7, AAV8, AAV9, AAV-NP66, AAV-NP22, AAV-NP94 vectors, or derivatives thereof. The methods employ known techniques of library generation; however, the methods are novel in that they employ replication competent AAV vectors during the variant capsid polypeptide generation (i.e., selection and evolution of the variant capsid polypeptides). The present invention provides methods for generating variant AAV capsid polypeptides, wherein the variant capsid polypeptides exhibit increased transduction or tropism in human liver or pancreatic tissue or cells as compared to non-variant parent capsid polypeptides, the method comprising: (a) generating a library of variant capsid polypeptide genes, wherein said variant capsid polypeptide genes include a plurality of variant capsid polypeptide genes comprising sequences from more than one non-variant parent capsid polypeptide; (b) generating an AAV vector library by cloning said variant capsid polypeptide gene library into AAV vectors, wherein said AAV vectors are replication competent AAV vectors; (c) screening said AAV vector library from (b) for variant capsid polypeptides exhibiting increased transduction or tropism in human liver or pancreatic tissue or cells as compared to a non-variant parent capsid polypeptide; and (d) selecting said variant AAV capsid polypeptides from (c). In some embodiments, the method further comprises (e) determining the sequence of said variant capsid polypeptides from (d).

In some embodiments, the variant AAV capsid polypeptides generated by screening methods of the invention exhibit increased transduction in human liver tissue or cells as compared to a vector encoding a non-variant parent capsid polypeptide. In some embodiments, the variant AAV capsid polypeptides generated by screening methods of the invention exhibit increased transduction in human pancreatic tissue or cells as compared to a vector encoding a non-variant parent capsid polypeptide. In some embodiments, transduction is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction is increased by about 5% to about 80%, about 10% to about 70%, about 20% to about 60%, about 30% to about 60%, or about 40% to about 50%.

In some embodiments, the variant AAV capsid polypeptide generated by screening methods of the invention exhibits increased tropism in human liver tissue or cells as compared to a vector encoding a non-variant parent capsid. In some embodiments, the variant AAV capsid polypeptide generated by screening methods of the invention exhibits increased tropism in human pancreatic tissue or cells as compared to a vector encoding a non-variant parent capsid. In some embodiments, tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, tropism is increased by about 5% to about 80%, about 10% to about 70%, about 20% to about 60%, about 30% to about 60%, or about 40% to about 50%.

In some embodiments, the variant AAV capsid polypeptide generated by screening methods of the invention further exhibits an enhanced neutralization profile as compared to a vector encoding a non-variant parent capsid polypeptide. In some embodiments, the neutralization profile is enhanced by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, the neutralization profile is enhanced by about 5% to about 80%, about 10% to about 70%, about 20% to about 60%, about 30% to about 60%, or about 40% to about 50%. In some embodiments, an enhanced neutralization profile is determined by a reduction in the generation of neutralizing antibodies in a host. In some embodiments, the reduction in generation of neutralizing antibodies is a reduction of about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, the reduction in generation of neutralizing antibodies is a reduction of about 5% to about 80%, about 10% to about 70%, about 20% to about 60%, about 30% to about 60%, or about 40% to about 50%.

In some embodiments, the variant AAV capsid polypeptide generated by screening methods of the invention further exhibits increased transduction or tropism in one or more non-liver human tissues as compared to a vector encoding a non-variant parent capsid polypeptide. In some embodiments, the variant capsid polypeptide further exhibits increased transduction in one or more non-liver human tissues as compared to a vector encoding a non-variant parent capsid polypeptide. In some embodiments, the variant capsid polypeptide further exhibits increased tropism in one or more non-liver human tissues as compared to a vector encoding a non-variant parent capsid polypeptide. In some embodiments, transduction and/or tropism is increased by about 5%, about 10%, about 15%, about 20%, about 25%, about 30%, about 35%, about 40%, about 45%, about 50%, about 55%, about 60%, 65%, about 70%%, about 75%, about 80%, about 85%, about 90%, about 95%, about 99%, or about 100%. In some embodiments, transduction and/or tropism is increased by about 5% to about 80%, about 10% to about 70%, about 20% to about 60% or about 30% to about 60%.

In some embodiments, the variant AAV capsid polypeptide generated by screening methods of the invention further exhibits increased transduction or tropism in one or more non-liver human tissues as compared to a vector encoding a non-variant parent capsid polypeptide. In some embodiments, the variant capsid polypeptide further exhibits increased transduction in one or more non-liver human tissues as compared to a vector encoding a non-variant parent capsid polypeptide. In some embodiments, the variant capsid polypeptide further exhibits increased tropism in one or more non-liver human tissues as compared to a vector encoding a non-variant parent capsid polypeptide. In some embodiments, transduction and/or tropism is increased by about 5%, about 10%, about 15%, about 20%, about 25%, about 30%, about 35%, about 40%, about 45%, about 50%, about 55%, about 60%, 65%, about 70%, about 75%, about 80%, about 85%, about 90%, about 95%, about 99%, or about 100%. In some embodiments, transduction and/or tropism is increased by about 5% to about 80%, about 10% to about 70%, about 20% to about 60% or about 30% to about 60%.

Transduction can be measured by techniques known in the art, including, for example, immunohistochemical analysis, as well as other methods known in the art. In vitro transduction analysis can be performed in human liver stem cells, human pancreatic stem cells, human liver cells, human pancreatic cells, mouse liver cells, and mouse pancreatic cells, including for example by measuring GFP expression (or another marker gene) in order to determine transduction. In vivo or ex vivo transduction analysis can be measured by techniques known in the art, including, for example, firefly luciferase-based assays, again as described in the art, including for example by measuring luciferase expression (or another marker gene) in order to determine transduction. In some embodiments, marker expression from an AAV vector packaged with the variant capsid polypeptides is compared to marker expression from an AAV vector packaged with the non-variant parent capsid polypeptides in order to determine changes in transduction efficiency. In some embodiments, the transduction is compared for different cell types in order to determine tropism, i.e., compare transduction from an AAV vector packaged with the variant capsid polypeptide to transduction from an AAV packaged with the non-variant capsid polypeptide in at least two different cell types in order to determine tropism for a particular cell type, sometimes referred to as a tropism profile. In some embodiments, the at least one cell type is human pancreatic tissue or human liver cells, including CD200-positive cells. In some embodiments, the at least one cell type is human liver tissue or human liver cells, including ASGPR-positive cells. In some embodiments, at least a second cell type includes but is not limited to blood cells, blood stem cells, muscle cells, gonads, germ cells, joint tissue or cells, pancreas (including (3-islet cells), spleen tissue or cells, the gastrointestinal tract (e.g., epithelium and/or smooth muscle), lung tissue or cells and/or kidney tissue or cells.

Such methods for generating the variant capsid polypeptides include DNA shuffling of capsid proteins, which begins with families of capsid genes from an array or plurality of AAV pseudo-species (for example, AAV-DJ, AAV1, AAV2, AAV3b, AAV4, AAV5, AAV6, AAV7, AAV8, AAV9, AAV 9 hu14, bovine AAV, avian AAV), that are enzymatically shuffled to create a diverse library of capsid genes that can be cloned back into an AAV shuttle plasmid and utilized to produce live replicating viral libraries. To maximize the likelihood that shuffled capsids (i.e., variant capsid polypeptides) could functionally transduce human liver tissue or liver cells—as compared to liver tissue or cells of model organisms typically used for pre-clinical evaluation—the invention contemplates performing screens using ASGPR-positive cells such as primary liver cells or CHO cells expressing ASGPR. In other cases, to maximize the likelihood that shuffled capsids could functionally transduce human pancreatic tissue or pancreatic cells, the invention contemplates performing screens using CD200-positive cells such as primary pancreatic cells or CHO cells expressing CD200.

In some embodiments, surgical liver or pancreatic biopsies from human patients are digested and purified by fluorescent activated cell sorting (FACS) to isolate a defined cell population that is ASGPR+(liver) or CD200+(pancreas) for use in screening. These cell populations can be maintained and cultured in stem cell/progenitor state or differentiated in short-term cultures, each with highly defined media. In some embodiments, cells of each type are pooled at equal ratios from the human patient biopsies to maximize cellular/patient variety, and replicating screens and carried for a plurality of rounds of selection (e.g., 2, 3, 4, 5, 6, or more rounds of selection) with diversity monitoring by sequencing beginning at, for example, round 3 and each round thereafter until the end of the screens. In some embodiments, characterization of the selected shuffled capsid variant sequences demonstrates diverse parental contribution.

At the completion of both screens, variants are chosen from each screened human cell type for full Sanger sequencing and phylogenetic comparisons to parental serotypes (i.e., parental non-variant capsid polypeptide sequences). In some embodiments, the parental non-variant capsid polypeptide sequences are those that went into the initial library. The most highly selected variants (for example, those that exhibit the highest increase in transduction and/or tropism) from each screen are isolated and vectorized with expression constructs, in some cases for use in subsequent validation experiments. In some embodiments, in order to assess the genetic contribution of each parental AAV serotype (i.e., non-variant parent capsid polypeptide) to the evolved capsids (i.e., variant capsid polypeptides) selected from each screen, crossover mapping can be performed and bioinformatic prediction analyses to calculate enrichment scores for likelihood of parental (i.e., non-variant parent capsid polypeptide) contribution to each position in the new capsids (i.e., variant capsid polypeptides). Both methodologies demonstrated the highly shuffled nature of the evolved capsid variants and highlighted both unique and shared domains present in selected capsids. In some embodiments, the parental capsids (i.e., non-variant parent capsid polypeptides) that contribute the most to the evolved variants include AAV-DJ, AAV1, AAV2, AAV3b, AAV8, and AAV9_hu14. In some embodiments, the variant capsid polypeptides comprise regions from AAV-DJ. AAV1, AAV2, AAV3b, AAV8, and AAV9_hu14. In some embodiments, no variants (i.e., variant capsid polypeptide) have capsid fragment regions from AAV4, 5, bovine or avian. In some embodiments, diverse shuffling was achieved and maintained along the length of Cap, including VP1, VP2 and VP3.

In vitro characterizations are used to demonstrate the significant increase in transduction by variant capsid polypeptides over control serotypes (i.e., non-variant parent capsid polypeptides) in various liver-derived cell lines or pancreas-derived cell lines.

For such analyses, large-scale ultrapure productions of AAV vectorized variants (AAV vectors composed of variant capsid polypeptides) can be carried out and those capable of producing high titers sufficient for eventual clinical use (for example, variants NP6, NP22, NP36, NP66, NP81 and NP94) are considered further for validation. In some embodiments, further validation includes FLuc transduction efficiency assessments in vitro in human hepatocytes with comparisons to the current best liver-tropic AAV serotypes 1, 6 and 8. In some embodiments, in primary human hepatocytes, shuffled variants (i.e., variant capsid polypeptides) exhibiting significantly increased functional transduction over vectors encoding AAV1, AAV6, and/or AAV8 non-variant parent capsids, by luciferase assay, can be selected by the present invention. In some embodiments, in human hepatocytes, shuffled variants (i.e., variant capsid polypeptides) exhibiting significantly increased functional transduction over AAV1, AAV6, and/or AAV8, can be selected by the present invention. A similar approach can be carried out using human pancreatic cells.

In order to examine the activity of the AAV vectors encoding the variant capsid polypeptides of the invention, in one embodiment, chimeric humanized liver xenografts are employed. Chimeric humanized liver xenografts are a powerful surrogate for assessing potential liver transduction in humans in vivo. To more rigorously assess the transduction capabilities of shuffled (i.e., variant capsid polypeptides) and control AAV capsids (i.e., non-variant parent capsid polypeptides) in an in vivo setting, variants can be characterized using chimeric humanized liver mouse xenograft model (as described in Strom et al., Methods Mol Biol, 2010, 640:491-509; incorporated by reference herein for all purposes). In this model, the human liver cells in the mouse host can exhibit metabolic pathways normally expressed in human liver. Large cohorts of xenografted mice can be produced and then administered shuffled (i.e., AAV vectors encoding variant parent capsid polypeptides) or control rAAV variants (i.e., AA vectors encoding variant parent capsid polypeptides) by direct injection (e.g., intramuscular injection) and assessed for transduction in time-course studies, including studies for 1-month, 2-months, 3-months, 4-months, 5-months, or 6-months or more. In some embodiments, the AAV vector encoding a non-variant parent capsid polypeptide is the first vector to uncoat and express, but the AAV vector encoding a variant parent capsid polypeptide produces the highest sustained transduction levels. In some embodiments, the AAV vector encoding a non-variant parent capsid polypeptide exhibits decreasing expression with time. In some embodiments, transplantation efficiency and species-specific transduction are controlled for by simultaneous performance of the same transduction time course experiment in strain- and gender-matched non-transplanted immune-deficient mice. Alternatively, chimeric humanized pancreatic xenografts can be employed.

In order to examine the activity of the AAV vectors encoding the variant capsid polypeptides of the invention, further validation can be performed using ex vivo human liver or pancreas explants. Ex vivo human liver or pancreas explant transductions are employed to validate the significantly increased expression in human liver or pancreas tissue specifically.

While chimeric humanized liver or pancreas xenografts are powerful tools to model human-like in vivo systems, they are limited in their ability to truly define expected transduction in human patients given the continued presence of mouse cells. To overcome these limitations and more accurately predict tissue-specific transduction in human patients, transduction of live human liver or pancreas explants from surgical biopsies ex vivo is contemplated by the methods of the invention. In some cases, a liver tissue sample is obtained from the liver of subjects via surgical biopsy procedures and employed for ex vivo transduction analyses. In other cases, a pancreatic tissue sample is obtained from the subject via a surgical biopsy procedure. For such methods, tissue biopsies are digested and individual hepatocytes are isolated for 24-hours, 48-hours, or 72-hours in culture and AAV vectors are administered within 30-minutes, 1-hour, 2-hours, 3-hours or 4-hours of removal of the liver tissue from the subject. In some embodiments, the tissue biopsies are digested and individual cells isolated for 48-hours in culture and AAV vectors are administered within 1 hour of removal of the tissue from the subject. In some embodiments, ex vivo nonhuman primate tissue explant transduction can also be employed for further validation.

In some embodiments, increased transduction of AAV vectors encoding variant parent capsid polypeptide is exhibited in CD200 positive pancreatic islet cells. In some embodiments, increased transduction of AAV vectors encoding variant parent capsid polypeptide is exhibited in ASGPR positive parenchymal liver cells.

### AAV Vector Elements

The nucleic acid insert (also referred to as a heterologous nucleotide sequence) can be operably linked to control elements directing the transcription or expression thereof in the nucleotide sequence in vivo. Such control elements can comprise control sequences normally associated with the selected gene (e.g., endogenous cellular control elements). Alternatively, heterologous control sequences can be employed. Useful heterologous control sequences generally include those derived from sequences encoding mammalian or viral genes. Examples include, but are not limited to, the SV40 early promoter, mouse mammary tumor virus long terminal repeat (LTR) promoter; adenovirus major late promoter (Ad MLP); a herpes simplex virus (HSV) promoter, an endogenous cellular promoter heterologous to the gene of interest, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, can also be used. Such promoter sequences are commercially available from, e.g., Stratagene (San Diego, Calif.) or ThermoFisher (Carlsbad, Calif.).

In some embodiments, a cell type-specific or a tissue-specific promoter can be operably linked to nucleic acid insert (also referred to as a heterologous nucleotide sequence) encoding the heterologous gene product, and allowing for selectively or preferentially producing a gene product in a particular cell type(s) or tissue(s). In some embodiments, an inducible promoter can be operably linked to the heterologous nucleic acid.

In some embodiments, the nucleic acid is packaged with the variant capsid polypeptides of the invention. In some embodiments, the nucleic acid insert or packaged nucleic acid is at least 50, 100, 200, 300, 400, 500, 600, 700, 800, 900, 1000, 1100, 1200, 1300, 1400, or 1500 nucleic acids in length. In some embodiments, the nucleic acid insert or packaged nucleic acid is at least 50 nucleic acids to at least 1500 nucleic acids. In some embodiments, the nucleic acid insert or packaged nucleic acid is at least 100 nucleic acids to at least 1400 nucleic acids. In some embodiments, the nucleic acid insert or packaged nucleic acid is at least 200 nucleic acids to at least 1100 nucleic acids. In some embodiments, the nucleic acid insert or packaged nucleic acid is at least 300 nucleic acids to at least 1000 nucleic acids. In some embodiments, the nucleic acid insert or packaged nucleic acid is at least 100 nucleic acids to at least 900 nucleic acids. In some embodiments, the nucleic acid insert or packaged nucleic acid is at least 200 nucleic acids to at least 900 nucleic acids. In some embodiments, the nucleic acid insert or packaged nucleic acid is at least 300 nucleic acids to at least 900 nucleic acids. In some embodiments, the nucleic acid insert or packaged nucleic acid is at least 100 nucleic acids to at least 600 nucleic acids.

In some embodiments, the AAV vector packaged by the variant capsid polypeptides is at least about 2000 nucleic acids in total length and up to about 5000 nucleic acids in total length. In some embodiments, the AAV vector packaged by the variant capsid polypeptides is about 2000 nucleic acids, about 2400 nucleic acids, about 2800 nucleic acids, about 3000 nucleic acids, about 3200 nucleic acids, about 3400 nucleic acids, about 3600 nucleic acids, about 3800 nucleic acids, about 4000 nucleic acids, about 4200 nucleic acids, about 4400 nucleic acids, about 4600 nucleic acids, about 4700 nucleic acids, or about 4800 nucleic acids. In some embodiments, the AAV vector packaged by the variant capsid polypeptides is between about 2000 nucleic acids (2 kb) and about 5000 nucleic acids (5 kb). In some embodiments, the AAV vector packaged by the variant capsid polypeptides is between about 2400 nucleic acids (2.4 kb) and about 4800 nucleic acids (4.8 kb). In some embodiments, the AAV vector packaged by the variant capsid polypeptides is between about 3000 nucleic acids (3 kb) and about 5000 nucleic acids (5 kb). In some embodiments, the AAV vector packaged by the variant capsid polypeptides is between about 3000 nucleic acids (3 kb) and about 4000 nucleic acids (4 kb).

Purified infectious AAV virions contain three major structural proteins designated VP1, VP2, and VP3 (87, 73, and 62 kDa, respectively) in an approximate ratio of 1:1:8. In some embodiments, the AAV vector has portions of the AAV vector deleted, in order to allow for more space during AAV vector packaging into an AAV virion. In some embodiments, additional sequences are deleted from the AAV vector, including but not limited to the VP2 capsid proteins (a capsid protein not required for viral infectivity), as well as other portions of the AAV vector including those described herein. In some embodiments, the deleted sequences allow for increased volume in order to package AAV vectors with increased nucleic acid insert lengths into the AAV virion as described herein. In some embodiments, the deleted sequences allow for increased interaction with a positive charge in order to package AAV vectors with increased nucleic acid insert lengths into the AAV virion as described herein.

The AAV vectors or AAV virions disclosed herein can also include conventional control elements operably linked to the nucleic acid insert (also referred to as a heterologous nucleotide sequence) in a manner permitting transcription, translation and/or expression in a cell transfected with the AAV vector or infected with the AAV virion produced according to the present invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters selected from native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the beta-actin promoter, the phosphoglycerol kinase (PGK) promoter, and the EF1 promoter (Invitrogen). Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., (1996) Proc. Natl. Acad. Sci. USA, 93:3346-3351), the tetracycline-repressible system (Gossen et al., (1992) Proc. Natl. Acad. Sci. USA, 89:5547-5551), the tetracycline-inducible system (Gossen et al., (1995) Science, 268:1766-1769, see also Harvey et al., (1998) Curr. Opin. Chem. Biol., 2:512-518), the RU486-inducible system (Wang et al., (1997) Nat. Biotech., 15:239-243 and Wang et al., (1997) Gene Ther., 4:432-441) and the rapamycin-inducible system (Magari et al., (1997) J. Clin. Invest., 100:2865-2872). Other types of inducible promoters useful in this context are those regulated by a specific physiological state, e.g., temperature, acute phase, a particular differentiation state of the cell, or in replicating cells only.

In another embodiment, the native promoter for the nucleic acid insert (also referred to as a heterologous nucleotide sequence) will be used. The native promoter may be preferred when it is desired that expression of the nucleic acid insert (also referred to as a heterologous nucleotide sequence) should mimic the native expression. The native promoter may be used when expression of the nucleic acid insert (also referred to as a heterologous nucleotide sequence) must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the nucleic acid insert (also referred to as a heterologous nucleotide sequence) includes a gene operably linked to a tissue-specific promoter. For instance, if expression in liver is desired, a promoter active in liver should be used. Examples of promoters that are tissue-specific are known for liver including albumin promoter (Miyatake et al., (1997) J. Virol., 71:5124-32), hepatitis B virus core promoter (Sandig et al., (1996) Gene Ther., 3:1002-9), alpha-fetoprotein promoter (AFP) (Arbuthnot et al., (1996) Hum. Gene Ther., 7:1503-14), among others. In some cases, if expression in the pancreas is desired, a promoter active in the pancreas should be used. Exemplary pancreatic specific promoters are described in, for example, Magnuson and Osipovich, Cell Metabolism, 2013, 18(1), 9-20.

In various embodiments, AAV vectors or AAV virions carrying one or more therapeutically useful nucleic acid inserts (also referred to as a heterologous nucleotide sequence) also include selectable markers or reporter genes, e.g., sequences encoding geneticin, hygromycin or puromycin resistance, among others. Selectable reporters or marker genes can be used to signal the presence of the plasmids/vectors in bacterial cells, including, for example, examining ampicillin resistance. Other components of the plasmid may include an origin of replication. Selection of these and other promoters and vector elements are conventional and many such sequences are available (see, e.g., Sambrook et al., and references cited therein).

### Host Cells and Packaging

Host cells are necessary for generating infectious AAV vectors as well as for generating AAV virions based on the disclosed AAV vectors. Accordingly, the present invention provides host cells for generation and packaging of AAV virions based on the AAV vectors of the present invention. A variety of host cells are known in the art and find use in the methods of the present invention. Any host cells described herein or known in the art can be employed with the compositions and methods described herein.

The present invention provides host cells, e.g., isolated (genetically modified) host cells, comprising a subject nucleic acid. A subject host cell can be an isolated cell, e.g., a cell in in vitro culture. A subject host cell is useful for producing a subject AAV vector or AAV virion, as described below. Where a subject host cell is used to produce a subject AAV virion, it is referred to as a “packaging cell.” In some embodiments, a subject host cell is stably genetically modified with a subject AAV vector. In other embodiments, a subject host cell is transiently genetically modified with a subject AAV vector.

In some embodiments, a subject nucleic acid is introduced stably or transiently into a host cell, using established techniques, including, but not limited to, electroporation, calcium phosphate precipitation, liposome-mediated transfection, baculovirus infection, and the like. For stable transformation, a subject nucleic acid will generally further include a selectable marker, e.g., any of several well-known selectable markers such as neomycin resistance, and the like.

Generally, when delivering the AAV vector according to the present invention by transfection, the AAV vector is delivered in an amount from about 5 μg to about 100 μg DNA, about 10 to about 50 μg DNA to about 1×10⁴ cells to about 1×10¹³ cells, or about 1×10⁵ cells. However, the relative amounts of vector DNA to host cells may be adjusted, taking into consideration such factors as the selected vector, the delivery method and the host cells selected and such adjustments are within the level of skill of one in the art.

In some embodiments, the host cell for use in generating infectious virions can be selected from any biological organism, including prokaryotic (e.g., bacterial) cells, and eukaryotic cells, including, insect cells, yeast cells and mammalian cells. A subject host cell is generated by introducing a subject nucleic acid (i.e., AAV vector) into any of a variety of cells, e.g., mammalian cells, including, e.g., murine cells, and primate cells (e.g., human cells). Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, CHO, 293, Vero, NIH 3T3, PC12, Huh-7 Saos, C2C12, RAT1, Sf9, L cells, HT1080, human embryonic kidney (HEK), human embryonic stem cells, human adult tissue stem cells, pluripotent stem cells, induced pluripotent stem cells, reprogrammed stem cells, organoid stem cells, bone marrow stem cells, HLHepG2, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirement for the cell used is it is capable of infection or transfection by an AAV vector. In some embodiments, the host cell is one that has rep and cap stably transfected in the cell, including in some embodiments a variant capsid polypeptide as described herein. In some embodiments, the host cell expresses a variant capsid polypeptide of the invention or part of an AAV vector as described herein, such as a heterologous nucleic acid sequence contained within the AAV vector.

In some embodiments, the preparation of a host cell according to the invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods providing the desired nucleotide sequence.

In some embodiments, introduction of the AAV vector into the host cell may also be accomplished using techniques known to the skilled artisan and as discussed throughout the specification. In a preferred embodiment, standard transfection techniques are used, e.g., CaPO₄ transfection or electroporation, and/or infection by hybrid adenovirus/AAV vectors into cell lines such as the human embryonic kidney cell line HEK 293 (a human kidney cell line containing functional adenovirus E1 genes providing trans-acting E1 proteins).

In some embodiments, a subject genetically modified host cell includes, in addition to a nucleic acid comprising a nucleotide sequence encoding a variant AAV capsid protein, as described above, a nucleic acid that comprises a nucleotide sequence encoding one or more AAV rep (replication) proteins. In other embodiments, a subject host cell further comprises an AAV vector. An AAV virion can be generated using a subject host cell. Methods of generating an AAV virion are described in, e.g., U.S. Patent Publication No. 2005/0053922 and U.S. Patent Publication No. 2009/0202490.

In addition to the AAV vector, in exemplary embodiments, the host cell contains the sequences driving expression of the AAV capsid polypeptide (including variant capsid polypeptides and non-variant parent capsid polypeptides) in the host cell and rep (replication) sequences of the same serotype as the serotype of the AAV Inverted Terminal Repeats (ITRs) found in the nucleic acid insert (also referred to as a heterologous nucleotide sequence), or a cross-complementing serotype. The AAV capsid and rep (replication) sequences may be independently obtained from an AAV source and may be introduced into the host cell in any manner known to one of skill in the art or as described herein. Additionally, when pseudotyping an AAV vector in an AAV8 capsid for example, the sequences encoding each of the essential rep (replication) proteins may be supplied by AAV8, or the sequences encoding the rep (replication) proteins may be supplied by different AAV serotypes (e.g., AAV1, AAV2, AAV3, AAV4, AAV5, AAV6, AAV7, AAV9, and/or chimeric serotypes thereof).

In some embodiments, the host cell stably contains the capsid protein under the control of a suitable promoter (including, for example, the variant capsid polypeptides of the invention), such as those described above. In some embodiments, the capsid protein is expressed under the control of an inducible promoter. In some embodiments, the capsid protein is supplied to the host cell in trans. When delivered to the host cell in trans, the capsid protein may be delivered via a plasmid containing the sequences necessary to direct expression of the selected capsid protein in the host cell. In some embodiments, when delivered to the host cell in trans, the vector encoding the capsid protein (including, for example, the variant capsid polypeptides of the invention) also carries other sequences required for packaging the AAV, e.g., the rep (replication) sequences.

In some embodiments, the host cell stably contains the rep (replication) sequences under the control of a suitable promoter, such as those described above. In some embodiments, the essential rep (replication) proteins are expressed under the control of an inducible promoter. In another embodiment, the rep (replication) proteins are supplied to the host cell in trans. When delivered to the host cell in trans, the rep (replication) proteins may be delivered via a plasmid containing the sequences necessary to direct expression of the selected rep (replication) proteins in the host cell. In some embodiments, when delivered to the host cell in trans, the vector encoding the capsid protein (including, for example, the variant capsid polypeptides of the invention) also carries other sequences required for packaging the AAV vector, e.g., the rep (replication) sequences.

In some embodiments, the rep (replication) and capsid sequences may be transfected into the host cell on a single nucleic acid molecule and exist stably in the cell as an unintegrated episome. In another embodiment, the rep (replication) and capsid sequences are stably integrated into the chromosome of the cell. Another embodiment has the rep (replication) and capsid sequences transiently expressed in the host cell. For example, a useful nucleic acid molecule for such transfection comprises, from 5′ to 3′, a promoter, an optional spacer interposed between the promoter and the start site of the rep (replication) gene sequence, an AAV rep (replication) gene sequence, and an AAV capsid gene sequence.

Although the molecule(s) providing rep (replication) and capsid can exist in the host cell transiently (i.e., through transfection), in some embodiments, one or both of the rep (replication) and capsid proteins and the promoter(s) controlling their expression be stably expressed in the host cell, e.g., as an episome or by integration into the chromosome of the host cell. The methods employed for constructing embodiments of the invention are conventional genetic engineering or recombinant engineering techniques such as those described in the references above.

In some embodiments, the packaging host cell can require helper functions in order to package the AAV vector of the invention into an AAV virion. In some embodiments, these functions may be supplied by a herpesvirus. In some embodiments, the necessary helper functions are each provided from a human or non-human primate adenovirus source, and are available from a variety of sources, including the American Type Culture Collection (ATCC), Manassas, Va. (US). In some embodiments, the host cell is provided with and/or contains an E1a gene product, an E1b gene product, an E2a gene product, and/or an E4 ORF6 gene product. In some embodiments, the host cell may contain other adenoviral genes such as VAI RNA. In some embodiments, no other adenovirus genes or gene functions are present in the host cell.

### Heterologous Nucleic Acids, Nucleic Acid Gene Products and Polypeptide Gene Products

In various embodiments, the invention provides variant capsid polypeptides capable of forming capsids capable of packaging a variety of therapeutic molecules, including nucleic acids and polypeptides. In some embodiments, the therapeutic molecule is a vaccine. In various embodiments, the invention provides for AAV vectors capable of containing nucleic acid inserts, including for example, transgene inserts or other nucleic acid inserts. This allows for vectors capable of expressing polypeptides. Such nucleic acids can comprise heterologous nucleic acid, nucleic acid gene products, and polypeptide gene products. Features of the nucleic acid inserts are described below.

In some embodiments, the AAV vectors described herein contain nucleic acid inserts. In some embodiments, the nucleic acid insert includes but is not limited to nucleic acid sequences selected from the group consisting of a non-coding RNA, a protein coding sequence, an expression cassette, a multi-expression cassette, a sequence for homologous recombination, a genomic gene targeting cassette, and a therapeutic expression cassette.

In some embodiments, the expression cassette is a CRISPR/CAS expression system.

In some embodiments, a nucleic acid insert comprises a heterologous nucleic acid comprising a nucleotide sequence encoding a heterologous gene product, e.g., a nucleic acid gene product or a polypeptide gene product. In some embodiments, the gene product is an interfering RNA (e.g., shRNA, siRNA, miRNA). In some embodiments, the gene product is an aptamer. The gene product can be a self-complementary nucleic acid. In some embodiments, the gene product is a polypeptide.

Suitable heterologous gene product includes interfering RNA, antisense RNA, ribozymes, and aptamers. Where the gene product is an interfering RNA (RNAi), suitable RNAi include RNAi that decrease the level of a target polypeptide in a cell.

In some embodiments, useful therapeutic products encoded by the heterologous nucleic acid sequence include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful heterologous nucleic acid sequence products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factor alpha and tumor necrosis factor beta, interferon alpha, interferon beta, and interferon gamma, stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, used to reduce overexpression of a target.

In some embodiments, the present invention provides methods for treatment of a stem cell disorder, for example a disorder in either bone marrow stem cells or adult tissue stem cells (i.e., somatic stem cells). In some embodiments, adult stem cells can include organoid stem cells (i.e., stem cells derived from any organ or organ system of interest within the body). Organs of the body include for example but are not limited to skin, hair, nails, sense receptors, sweat gland, oil glands, bones, muscles, brain, spinal cord, nerve, pituitary gland, pineal gland, hypothalamus, thyroid gland, parathyroid, thymus, adrenals, pancreas (islet tissue), heart, blood vessels, lymph nodes, lymph vessels, thymus, spleen, tonsils, nose, pharynx, larynx, trachea, bronchi, lungs, mouth, pharynx, esophagus, stomach, small intestine, large intestine, rectum, anal canal, teeth, salivary glands, tongue, liver, gallbladder, pancreas, appendix, kidneys, ureters, urinary bladder, urethra, testes, ductus (vas) deferens, urethra, prostate, penis, scrotum, ovaries, uterus, uterine (fallopian) tubes, vagina, vulva, and mammary glands (breasts). Organ systems of the body include but are not limited to the integumentary system, skeletal system, muscular system, nervous system, endocrine system, cardiovascular system, lymphatic system, respiratory system, digestive system, urinary system, and reproductive system. In some embodiments, the disorder for treatment is a disorder in any one or more organoid stem cells (i.e., stem cells derived from any organ or organ system of interest within the body). In some embodiments, the treatment is in vivo (for example, administration of the variant capsid polypeptides is directly to the subject). In some embodiments, the treatment is ex vivo (for example, administration of the variant capsid polypeptides is to stem cells isolated from the subject and the treated stem cells are then returned to the subject).

### Methods for Generating an AAV Virion

In various embodiments, the invention provides a method for generating an AAV virion of the invention. A variety of methods of generating AAV virions are known in the art and can be used to generate AAV virions comprising the AAV vectors described herein. Generally, the methods involved inserting or transducing an AAV vector of the invention into a host cell capable of packaging the AAV vector into and AAV virion. Exemplary methods are described and referenced below; however, any method known to one of skill in the art can be employed to generate the AAV virions of the invention.

An AAV vector comprising a heterologous nucleic acid and used to generate an AAV virion can be constructed using methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, N.Y., Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Suitable host cells for producing AAV virions include microorganisms, yeast cells, insect cells, and mammalian cells, that can be, or have been, used as recipients of a heterologous DNA molecule. The term includes the progeny of the original cell transfected. Thus, a “host cell” as used herein generally refers to a cell transfected with an exogenous DNA sequence. Cells from the stable human cell line, 293 (readily available through, e.g., the American Type Culture Collection under Accession Number ATCC CRL1573) can be used. For example, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a convenient platform in which to produce AAV virions.

Methods of producing an AAV virion in insect cells are known in the art, and can be used to produce a subject AAV virion. See, e.g., U.S. Patent Publication No. 2009/0203071; U.S. Pat. No. 7,271,002; and Chen (2008) Mol. Ther. 16:924.

In some embodiments, the AAV virion or AAV vector is packaged into an infectious virion or virus particle, by any of the methods described herein or known in the art.

In some embodiments, the variant capsid polypeptide allows for similar packaging as compared to a non-variant parent capsid polypeptide.

In some embodiments, an AAV vector packaged with the variant capsid polypeptide is transduced into cells in vivo better than a vector packaged with a non-variant parent capsid polypeptide.

In some embodiments, the AAV vector packaged with the variant capsid polypeptide is transduced into cells in vitro better than a vector packaged with a non-variant parent capsid polypeptide.

In some embodiments, the variant capsid polypeptide results in nucleic acid expression higher than to a nucleic acid packaged with a non-variant parent capsid polypeptide.

In some embodiments, the AAV vector packaged with said variant capsid polypeptide results in transgene expression better than a transgene packaged with a non-variant parent capsid polypeptide.

### Pharmaceutical Compositions and Dosing

The present invention provides pharmaceutical compositions useful in treating subjects according to the methods of the invention as described herein. Further, the present invention provides dosing regimens for administering the described pharmaceutical compositions. The present invention provides pharmaceutical compositions comprising: a) a subject AAV vector or AAV virion, as described herein as well as therapeutic molecules packaged by or within capsids comprising variant polypeptides as described herein; and b) a pharmaceutically acceptable carrier, diluent, excipient, or buffer. In some embodiments, the pharmaceutically acceptable carrier, diluent, excipient, or buffer is suitable for use in a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A subject composition can comprise a liquid comprising a subject variant AAV capsid polypeptide of the invention or AAV virion comprising a variant capsid polypeptide in solution, in suspension, or both. As used herein, liquid compositions include gels. In some cases, the liquid composition is aqueous. In some embodiments, the composition is an in situ gellable aqueous composition, e.g., an in situ gellable aqueous solution. Aqueous compositions have opthalmically compatible pH and osmolality.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound. Preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Useful carriers include Vaseline®, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the AAV vector or AAV virion and methods and uses of are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

Methods and uses of the invention as disclosed herein can be practiced within about 1 hour to about 2 hours, about 2 hours to about 4 hours, about 4 hours to about 12 hours, about 12 hours to about 24 hours or about 24 hours to about 72 hours after a subject has been identified as having the disease targeted for treatment, has one or more symptoms of the disease, or has been screened and is identified as positive as set forth herein even though the subject does not have one or more symptoms of the disease. In some embodiments, the invention as disclosed herein can be practiced within about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 36 hours, about 48 hours, or about 72 hours or more. Of course, methods and uses of the invention can be practiced about 1 day to about 7 days, about 7 days to about 14 days, about 14 days to about 21 days, about 21 days to about 48 days or more, months or years after a subject has been identified as having the disease targeted for treatment, has one or more symptoms of the disease, or has been screened and is identified as positive as set forth herein. In some embodiments, the invention as disclosed herein can be practiced within about 1 day, about 2 days, about 3 days, about 4 days, about 5 days, about 6 days, about 7 days, about 8 days, about 9 days, about 10 days, about 11 days, about 12 days, about 14 days, about 21 days, about 36 days, or about 48 days or more.

In some embodiments, the present invention provides kits with packaging material and one or more components therein. A kit typically includes a label or packaging insert including a description of the components or instructions for use in vitro, in vivo, or ex vivo, of the components therein. A kit can contain a collection of such components, e.g., a variant AAV capsid polypeptide, an AAV vector, or AAV virion and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease a kit component may be used for. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another incompatible treatment protocol or therapeutic regimen and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

### Method of Treating a Disease

The present invention also provides methods for treatment of disease in a subject by administering the AAV vectors and/or nucleic acids of the present invention, wherein AAV vectors and/or nucleic acids described herein are packaged within a functional AAV capsid, and wherein the functional AAV capsid comprises one or more variant capsid polypeptides of the present invention. In an exemplary embodiment, the invention provides a method of administering a pharmaceutical composition of the invention to a subject in need thereof to treat a disease of a subject. In various embodiments, the subject is not otherwise in need of administration of a composition of the invention. In some embodiments, the invention provides methods for vaccine administration.

In some embodiments, the variant AAV capsid polypeptide packages a therapeutic expression cassette comprised of a heterologous nucleic acid comprising a nucleotide sequence encoding a heterologous gene product, such as for example a therapeutic protein or vaccine. In some embodiments, the AAV virion or AAV vector comprises a therapeutic expression cassette comprised of a heterologous nucleic acid comprising a nucleotide sequence encoding a heterologous gene product, such as for example a therapeutic protein or vaccine.

In some embodiments, the variant capsid polypeptides of the invention are employed as part of vaccine delivery. Vaccine delivery can include delivery of any of the therapeutic proteins as well as nucleic acids described herein. In some embodiments, variant capsid polypeptides of the invention are employed as part of a vaccine regimen and dosed according to the methods described herein.

In some embodiments, the variant AAV capsid polypeptides, the AAV virions, or AAV vectors of the invention are used in a therapeutic treatment regimen.

In some embodiments, the variant AAV capsid polypeptides, the AAV virions, or AAV vectors of the invention are used for therapeutic polypeptide production.

In some cases, a subject variant AAV capsid polypeptide or AAV vector, when introduced into the cells of a subject provides for high level production of the heterologous gene product packaged by the variant AAV capsid polypeptide or encoded by the AAV. For example, a heterologous polypeptide packaged by the variant AAV capsid polypeptide or encoded by the AAV can be produced at a level of from about 1 μg to about 50 μg or more.

In some cases, a subject variant AAV capsid polypeptide, AAV virion, or AAV vector, when introduced into a subject provides for production of the heterologous gene product packaged by the variant AAV capsid polypeptide or encoded by the AAV vector in at least about 10%, at least about 15%, at least about 20%, at least about 25%, at least about 30%, at least about 35%, at least about 40%, at least about 50% at least about 60%, at least about 70%, at least about 80%, or more than 80%, of the target cells.

In some embodiments, the present invention provides a method of treating a disease, the method comprising administering to an individual in need thereof an effective amount of a therapeutic molecule packaged by the variant AAV capsid polypeptide or subject AAV virion as described above.

A variant AAV capsid polypeptide or subject AAV virion can be administered systemically, regionally or locally, or by any route, for example, by injection, infusion, orally (e.g., ingestion or inhalation), or topically (e.g., transdermally). Such delivery and administration include intravenously, intramuscularly, intraperitoneally, intradermally, subcutaneously, intracavity, intracranially, transdermally (topical), parenterally, e.g. transmucosally or rectally. Exemplary administration and delivery routes include intravenous (i.v.), intraperitoneal (i.p.), intrarterial, intramuscular, parenteral, subcutaneous, intra-pleural, topical, dermal, intradermal, transdermal, parenterally, e.g. transmucosal, intra-cranial, intra-spinal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic.

In some cases, a therapeutically effective amount of a therapeutic molecule packaged by the variant AAV capsid polypeptide or a subject AAV virion is an amount that, when administered to an individual in one or more doses, is effective to slow the progression of the disease or disorder in the individual, or is effective to ameliorate symptoms. For example, a therapeutically effective amount of a therapeutic molecule packaged by the variant AAV capsid polypeptide or a subject AAV virion can be an amount that, when administered to an individual in one or more doses, is effective to slow the progression of the disease by at least about 15%, at least about 20%, at least about 25%, at least about 30%, at least about 40%, at least about 50%, at least about 60%, at least about 70%, at least about 80%, or more than about 80%, compared to the progression of the disease in the absence of treatment with the therapeutic molecule packaged by the variant AAV capsid polypeptide or the AAV virion.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

Improvement of clinical symptoms can also be monitored by one or more methods known to the art, and used as an indication of therapeutic effectiveness. Clinical symptoms may also be monitored by anatomical or physiological means, such as indirect ophthalmoscopy, fundus photography, fluorescein angiopathy, optical coherence tomography, electroretinography (full-field, multifocal, or other), external eye examination, slit lamp biomicroscopy, applanation tonometry, pachymetry, autorefaction, or other measures of functional vision. In some embodiments, a therapeutic molecule (including, for example, a vaccine) packaged by the variant AAV capsid polypeptide, a subject AAV virion, or AAV virus, when introduced into a subject, provides for production of the heterologous gene product for a period of time of from about 2 days to about 6 months, e.g., from about 2 days to about 7 days, from about 1 week to about 4 weeks, from about 1 month to about 2 months, or from about 2 months to about 6 months. In some embodiments, therapeutic molecule (including, for example, a vaccine) packaged by the variant AAV capsid polypeptide, a subject AAV virion or virus, when introduced into a subject provides for production of the heterologous gene product encoded for a period of time of more than 6 months, e.g., from about 6 months to 20 years or more, or greater than 1 year, e.g., from about 6 months to about 1 year, from about 1 year to about 2 years, from about 2 years to about 5 years, from about 5 years to about 10 years, from about 10 years to about 15 years, from about 15 years to about 20 years, or more than 20 years. In some embodiments, the administration regimen is part of a vaccination regimen.

Multiple doses of a subject AAV virion can be administered to an individual in need thereof. Where multiple doses are administered over a period of time, an active agent is administered once a month to about once a year, from about once a year to once every 2 years, from about once every 2 years to once every 5 years, or from about once every 5 years to about once every 10 years, over a period of time. For example, a subject AAV virion is administered over a period of from about 3 months to about 2 years, from about 2 years to about 5 years, from about 5 years to about 10 years, from about 10 years to about 20 years, or more than 20 years. The actual frequency of administration, and the actual duration of treatment, depends on various factors. In some embodiments, the administration regimen is part of a vaccination regimen.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can readily determine a virion dose range to treat a patient having a particular disease or disorder based on the aforementioned factors, as well as other factors. Generally, doses will range from at least about, or more, for example, about 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect.

In some embodiments, the variant AAV polypeptides of the present invention can be employed to reduce the amount of total AAV vector or other therapeutic molecule administered to a subject, wherein less total AAV vector or other therapeutic molecule is administered to a subject when said AAV vector or other therapeutic molecule is transduced using a variant capsid polypeptide as compared to the amount of AAV vector or other therapeutic molecule administered to a subject when the AAV vector or other therapeutic molecule is transduced using a non-variant parent capsid polypeptide in order to obtain a similar therapeutic effect (i.e., both dosages induce similar therapeutic effects or indistinguishable therapeutic effects). In some embodiments, the total vector or other therapeutic molecule administered to a subject is reduced by about 5%, about 10%, about 15%, about 20%, about 25%, about 30%, about 35%, about 40%, about 50%, about 55%, about 60%, about 65%, about 70%, about 75%, about 80% or more when an AAV vector or other therapeutic molecule is transduced using a variant capsid polypeptide as compared to when an AAV vector or other therapeutic molecule is transduced using a non-variant parent capsid polypeptide in order to obtain a similar therapeutic effect (i.e., both dosages induce similar therapeutic effects or indistinguishable therapeutic effects). In some embodiments, the total AAV vector or other therapeutic molecule administered to a subject is reduced by about 5% to about 80%, about 10% to about 75%, about 15% to about 65%, about 20% to about 60%, or about 10% to about 50% when the AAV vector or other therapeutic molecule is transduced using a variant capsid polypeptide as compared to when the AAV vector or other therapeutic molecule is transduced using a non-variant parent capsid polypeptide in order to obtain a similar therapeutic effect (i.e., both dosages induce similar therapeutic effects or indistinguishable therapeutic effects).

An effective amount or a sufficient amount can, but need not be, provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant clotting factor protein for treatment of a clotting disorder (e.g., hemophilia A or B).

An effective amount or a sufficient amount need not be effective in each and every subject treated, or a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

With regard to a disease or symptom thereof, or an underlying cellular response, a detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

Thus, a successful treatment outcome can lead to a “therapeutic effect,” or “benefit” of decreasing, reducing, inhibiting, suppressing, limiting, controlling or preventing the occurrence, frequency, severity, progression, or duration of a disease, or one or more adverse symptoms or underlying causes or consequences of the disease in a subject. Treatment methods and uses affecting one or more underlying causes of the disease or adverse symptoms are therefore considered to be beneficial. A decrease or reduction in worsening, such as stabilizing the disease, or an adverse symptom thereof, is also a successful treatment outcome.

A therapeutic benefit or improvement therefore need not be complete ablation of the disease, or any one, most or all adverse symptoms, complications, consequences or underlying causes associated with the disease. Thus, a satisfactory endpoint is achieved when there is an incremental improvement in a subject's disease, or a partial decrease, reduction, inhibition, suppression, limit, control or prevention in the occurrence, frequency, severity, progression, or duration, or inhibition or reversal, of the disease (e.g., stabilizing one or more symptoms or complications), over a short or long duration of time (hours, days, weeks, months, etc.). Effectiveness of a method or use, such as a treatment that provides a potential therapeutic benefit or improvement of a disease, can be ascertained by various methods.

Disclosed methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the invention, for example, a therapeutic method of treating a subject for a blood clotting disease.

The compound, agent, drug, treatment or other therapeutic regimen or protocol can be administered as a combination composition, or administered separately, such as concurrently or in series or sequentially (prior to or following) delivery or administration of an AAV vector or AAV virion as described herein. The invention therefore provides combinations where a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of an AAV vector or AAV virion as described herein, to a subject. Specific non-limiting examples of combination embodiments therefore include the foregoing or other compound, agent, drug, therapeutic regimen, treatment protocol, process, remedy or composition.

Methods and uses of the invention also include, among other things, methods and uses that result in a reduced need or use of another compound, agent, drug, therapeutic regimen, treatment protocol, process, or remedy. For example, for a blood clotting disease, a method or use of the invention has a therapeutic benefit if in a given subject a less frequent or reduced dose or elimination of administration of a recombinant clotting factor protein to supplement for the deficient or defective (abnormal or mutant) endogenous clotting factor in the subject. Thus, in accordance with the invention, methods and uses of reducing need or use of another treatment or therapy are provided.

The invention is useful in animals including veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

Non-limiting particular examples of diseases treatable in accordance with the invention include those set forth herein as well as a liver disease, a genetic disease of the liver, an acquired liver disease, type 1 or type 2 diabetes, liver cancer, a pancreatic disease, pancreatic cancer, pancreatitis, a lung disease (e.g., cystic fibrosis), a blood coagulation or bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, lysosomal storage diseases, a copper or iron accumulation disorders (e.g., Wilson's or Menkes disease) lysosomal acid lipase deficiency, a neurological or neurodegenerative disorder, cancer, Gaucher's disease, Hurler's disease, adenosine deaminase deficiency, a metabolic defect (e.g., glycogen storage diseases), a retinal degenerative disease (such as RPE65 deficiency or defect, choroideremia, and other diseases of the eye), and a disease of a solid organ (e.g., brain, liver, kidney, heart), as well as muscle diseases including not limited to Acid Maltase Deficiency (AMD), Amyotrophic Lateral Sclerosis (ALS), Andersen-Tawil Syndrome, Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

Ocular diseases that can be treated or prevented using a subject method include, but are not limited to, selected from acute macular neuroretinopathy; macular telangiectasia; Behcet's disease; choroidal neovascularization; diabetic uveitis; histoplasmosis; macular degeneration, such as acute macular degeneration, Scorsby's macular dystrophy, early or intermediate (dry) macular degeneration, or a form of advanced macular degeneration, such as exudative macular degeneration or geographic atrophy; edema, such as macular edema, cystoid macular edema and diabetic macular edema; multifocal choroiditis; ocular trauma affecting a posterior ocular site or location; ocular tumors; retinal disorders, such as central retinal vein occlusion, diabetic retinopathy (including proliferative and non-proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy; epiretinal membrane disorders; central or branch retinal vein occlusion; anterior ischemic optic neuropathy, non-retinopathy diabetic retinal dysfunction; retinitis pigmentosa; retinoschisis; and glaucoma.

In one embodiment, a method or use of the invention includes: (a) providing an AAV virion whose capsid comprises the variant AAV capsid polypeptides prepared as described herein, wherein the AAV virion comprises a heterologous nucleic acid sequence, wherein the heterologous nucleic acid sequence is operably linked to an expression control element conferring transcription of said nucleic acid sequence; and (b) administering an amount of the AAV virion to the mammal such that said heterologous nucleic acid is expressed in the mammal.

In one embodiment, a method or use of the invention includes: (a) providing a therapeutic molecule (including, for example, a vaccine) packaged by variant AAV capsid polypeptides prepared as described herein, wherein the therapeutic molecule comprises a heterologous nucleic acid sequence, wherein the heterologous nucleic acid sequence is operably linked to an expression control element conferring transcription of said nucleic acid sequence; and (b) administering an amount of the therapeutic molecule (including, for example, a vaccine) packaged by variant AAV capsid polypeptides to the mammal such that said heterologous nucleic acid is expressed in the mammal.

In another embodiment, a method or use of the invention includes delivering or transferring a heterologous polynucleotide sequence into a mammal or a cell of a mammal, by administering a heterologous polynucleotide packaged by a variant AAV capsid polypeptide, a plurality of heterologous polynucleotides packaged by variant AAV capsid polypeptides, an AAV virion prepared as described herein, or a plurality of AAV virions comprising the heterologous nucleic acid sequence to a mammal or a cell of a mammal, thereby delivering or transferring the heterologous polynucleotide sequence into the mammal or cell of the mammal. In some embodiments, the heterologous nucleic acid sequence encodes a protein expressed in the mammal, or where the heterologous nucleic acid sequence encodes an inhibitory sequence or protein that reduces expression of an endogenous protein in the mammal.

By way of example, respecting hemophilia, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than about 5% of normal is needed. With respect to treating such a hemophilic subject, a typical dose is at least 1×10¹⁰ AAV vector genomes (vg) per kilogram (vg/kg) of the weight of the subject, or between about 1×10¹⁰ to about 1×10¹¹ vg/kg of the weight of the subject, or between about 1×10¹¹ to about 1×10¹² vg/kg of the weight of the subject, or between about 1×10¹² to about 1×10¹³ vg/kg of the weight of the subject, to achieve a desired therapeutic effect.

### DETAILED DESCRIPTION OF EMBODIMENTS

The present invention provides a variant adeno-associated virus (AAV) capsid polypeptide comprising a designed ankyrin repeat protein (DARPin) or fragment thereof fused to the N-terminus of an AAV capsid protein VP2, wherein the DARPin specifically binds to a cell surface molecule expressed on human liver tissue or cells. In some embodiments, the variant capsid polypeptide described herein exhibits increased transduction or tropism in human liver tissue or cells as compared to a non-variant parent capsid polypeptide.

In some embodiments, the variant capsid polypeptide exhibits increased transduction as compared to a non-variant parent capsid polypeptide. In certain embodiments, the variant capsid polypeptide further exhibits an enhanced neutralization profile as compared to a non-variant parent capsid polypeptide. In other embodiments, the variant capsid polypeptide further exhibits increased transduction or tropism in one or more non-liver human tissues as compared to a non-variant parent capsid polypeptide.

In some embodiments, the variant capsid polypeptide exhibits increased transduction of human liver tissue or cells in vivo as compared to a non-variant parent capsid polypeptide. In certain embodiments, the variant capsid polypeptide exhibits increased transduction of human liver tissue or cells in vitro as compared to a non-variant parent capsid polypeptide. In other embodiments, the variant capsid polypeptide exhibits increased transduction of a human liver tissue explant ex vivo as compared to a non-variant parent capsid polypeptide.

In some embodiments, the cell surface molecule expressed on human liver tissue or cells is selected from the group consisting of asialoglycoprotein receptor (ASGPR).

The present invention also provides a variant adeno-associated virus (AAV) capsid polypeptide comprising a designed ankyrin repeat protein (DARPin) or fragment thereof fused to the N-terminus of an AAV capsid protein VP2, wherein the DARPin specifically binds to a cell surface molecule expressed on human pancreatic tissue or cells. The variant capsid polypeptide exhibits increased transduction or tropism in human pancreatic tissue or cells as compared to a non-variant parent capsid polypeptide.

In some embodiments, the variant capsid polypeptide exhibits increased transduction as compared to a non-variant parent capsid polypeptide. In certain embodiments, the variant capsid polypeptide further exhibits an enhanced neutralization profile as compared to a non-variant parent capsid polypeptide. In other embodiments, the variant capsid polypeptide further exhibits increased transduction or tropism in one or more non-pancreatic human tissues as compared to a non-variant parent capsid polypeptide.

In some embodiments, the variant capsid polypeptide exhibits increased transduction of human pancreatic tissue or cells in vivo as compared to a non-variant parent capsid polypeptide. In certain embodiments, the variant capsid polypeptide exhibits increased transduction of human pancreatic tissue or cells in vitro as compared to a non-variant parent capsid polypeptide. In other embodiments, the variant capsid polypeptide exhibits increased transduction of a human pancreatic tissue explant ex vivo as compared to a non-variant parent capsid polypeptide.

In some embodiments, the cell surface molecule expressed on human pancreatic tissue or cells is CD200. In certain embodiments, the variant capsid polypeptide comprises an amino acid sequence having at least 85%, at least 90%, at least about 95%, at least about 98%, or at least about 99%, or 100% amino acid sequence identity to the amino acid sequence selected from the group consisting of SEQ ID NOS: 1 to 4. In certain embodiments, the variant capsid polypeptide comprises an amino acid sequence selected from the group consisting of SEQ ID NOS: 1 to 4.

Any one of the variant capsid polypeptides described herein can be part of a functional AAV capsid. In certain embodiments, the functional AAV capsid packages a nucleic acid sequence selected from the group consisting of a non-coding RNA, a protein coding sequence, an expression cassette, a multi-expression cassette, a sequence for homologous recombination, a genomic gene targeting cassette, and a therapeutic expression cassette. The nucleic acid sequence can be contained within an AAV vector. Optionally, expression cassette is a CRISPR/CAS expression system. In some instances, the therapeutic expression cassette encodes a therapeutic protein or antibody.

Also provided are methods of using any variant AAV capsid polypeptide outlined herein in a therapeutic treatment regimen or vaccine. In some embodiments, the method can reduce the amount of total nucleic acid administered to a subject. The method can include administering less total nucleic acid amount to the subject when the nucleic acid is transduced using a variant capsid polypeptide as compared to the amount of nucleic acid administered to the subject when said nucleic acid is transduced using a non-variant parent capsid polypeptide in order to obtain a similar therapeutic effect.

The present invention provides an adeno-associated virus (AAV) vector comprising a nucleic acid sequence encoding a variant capsid polypeptide comprising a designed ankyrin repeat protein (DARPin) or fragment thereof fused to the N-terminus of an AAV capsid protein VP2. The DARPin specifically binds to a cell surface molecule expressed on human liver tissue or cells. In some embodiments, the variant capsid polypeptide exhibits increased transduction or tropism in human liver tissue or cells as compared to a non-variant parent capsid polypeptide.

In some embodiments, the variant capsid polypeptide exhibits increased transduction as compared to a non-variant parent capsid polypeptide. In certain embodiments, the variant capsid polypeptide further exhibits an enhanced neutralization profile as compared to a non-variant parent capsid polypeptide. In other embodiments, the variant capsid polypeptide further exhibits increased transduction or tropism in one or more non-liver human tissues as compared to a non-variant parent capsid polypeptide.

In some embodiments, the variant capsid polypeptide exhibits increased transduction of human liver tissue or cells in vivo as compared to a non-variant parent capsid polypeptide. In certain embodiments, the variant capsid polypeptide exhibits increased transduction of human liver tissue or cells in vitro as compared to a non-variant parent capsid polypeptide. In other embodiments, the variant capsid polypeptide exhibits increased transduction of a human liver tissue explant ex vivo as compared to a non-variant parent capsid polypeptide.

In some embodiments, the cell surface molecule expressed on human liver tissue or cells can be asialoglycoprotein receptor (ASGPR).

The present invention also provides an adeno-associated virus (AAV) vector containing a nucleic acid sequence encoding a variant capsid polypeptide comprising a designed ankyrin repeat protein (DARPin) or fragment thereof fused to the N-terminus of an AAV capsid protein VP2. In some instances, the DARPin specifically binds to a cell surface molecule expressed on human pancreatic tissue or cells. In some cases, the cell surface molecule expressed on the human pancreatic tissue or cells is CD200. In certain embodiments, the variant capsid polypeptide comprises an amino acid sequence selected from the group consisting of SEQ ID NOs:1 to 4. In some embodiments, the variant capsid polypeptide comprises an amino acid sequence having at least 85% (e.g., 85%, 86%, 87%, 89%, 90%, 91%, 92%, 93%, 94%, 95%, 96%, 97%, 98%, 99%, 99.%, or more) sequence identity to SEQ ID NO:1. In some embodiments, the variant capsid polypeptide comprises an amino acid sequence having at least 85% (e.g., 85%, 86%, 87%, 89%, 90%, 91%, 92%, 93%, 94%, 95%, 96%, 97%, 98%, 99%, 99.%, or more) sequence identity to SEQ ID NO:2. In some embodiments, the variant capsid polypeptide comprises an amino acid sequence having at least 85% (e.g., 85%, 86%, 87%, 89%, 90%, 91%, 92%, 93%, 94%, 95%, 96%, 97%, 98%, 99%, 99.%, or more) sequence identity to SEQ ID NO:3. In some embodiments, the variant capsid polypeptide comprises an amino acid sequence having at least 90% (e.g., 85%, 86%, 87%, 89%, 90%, 91%, 92%, 93%, 94%, 95%, 96%, 97%, 98%, 99%, 99%, or more) sequence identity to SEQ ID NO:4.

In some embodiments, variant capsid polypeptide exhibits increased transduction or tropism in human pancreatic tissue or cells as compared to a non-variant parent capsid polypeptide.

In some embodiments, the variant capsid polypeptide exhibits increased transduction as compared to a non-variant parent capsid polypeptide. In certain embodiments, the variant capsid polypeptide further exhibits an enhanced neutralization profile as compared to a non-variant parent capsid polypeptide. In other embodiments, the variant capsid polypeptide further exhibits increased transduction or tropism in one or more non-pancreatic human tissues as compared to a non-variant parent capsid polypeptide.

In some embodiments, the variant capsid polypeptide exhibits increased transduction of human pancreatic tissue or cells in vivo as compared to a non-variant parent capsid polypeptide. In certain embodiments, the variant capsid polypeptide exhibits increased transduction of human pancreatic tissue or cells in vitro as compared to a non-variant parent capsid polypeptide. In other embodiments, the variant capsid polypeptide exhibits increased transduction of a human pancreatic tissue explant ex vivo as compared to a non-variant parent capsid polypeptide.

In some embodiments, the AAV vector further includes a nucleic acid sequence selected from the group consisting of a non-coding RNA, a coding sequence, an expression cassette, a multi-expression cassette, a sequence for homologous recombination, a genomic gene targeting cassette, and a therapeutic expression cassette. The variant capsid polypeptide allows for nucleic acid expression similarly to a non-variant parent capsid polypeptide. In some instances, the expression cassette is a CRISPR/CAS expression system. In some instances, the therapeutic expression cassette encodes a therapeutic protein or antibody.

In one aspect, the present invention describes a method of using any of the AAV vectors disclosed herein in a therapeutic treatment regimen or vaccine. In another aspect, a method of using any of the AAV vectors to reduce the amount of total AAV vector administered to a subject is provided herein. The method can include administering less total AAV vector amount to the subject when the AAV vector is transduced by a variant capsid polypeptide, as compared to the amount of AAV vector administered to the subject when the AAV vector is transduced by a non-variant parent capsid polypeptide in order to obtain a similar therapeutic effect.

In some aspects, the present invention provides methods for generating a variant AAV capsid polypeptide comprising a designed ankyrin repeat protein (DARPin) or fragment thereof fused to the N-terminus of an AAV capsid protein VP2, wherein the DARPin specifically binds to a cell surface molecule expressed on human liver or pancreatic tissue or cells, wherein the variant capsid polypeptide exhibits increased transduction or tropism in human liver or pancreatic tissue or cells as compared to a non-variant parent capsid polypeptide. The method includes: (a) generating a library of variant capsid polypeptide genes, wherein said variant capsid polypeptide genes include a plurality of variant capsid polypeptide genes comprising sequences from more than one non-variant parent capsid polypeptide; (b) generating an AAV vector library by cloning said variant capsid polypeptide gene library into AAV vectors, wherein said AAV vectors are replication competent AAV vectors; (c) screening said AAV vector library from b) for variant AAV capsid polypeptides for increased transduction or tropism in human liver and pancreatic tissue or cells as compared to a non-variant parent capsid polypeptide; and (d) selecting said variant AAV capsid polypeptides from (c). In some embodiments, the method also includes performing (c) and (d) one or more times. In certain embodiments, the method further includes (e) determining the sequence of said variant capsid polypeptides from (d).

In some embodiments, the method also includes transducing target cells with the AAV vector library. The target cells can be recombinant cells expressing a cell surface molecule expressed on human liver or pancreatic tissue or cells. The cell surface molecule expressed on human liver tissue or cells can be ASGPR. The cell surface molecule expressed on human pancreatic tissue or cells can be CD200.

In some embodiments, the variant capsid polypeptide exhibits increased transduction as compared to a non-variant parent capsid polypeptide. In some embodiments, the variant capsid polypeptide exhibits increased tropism as compared to a non-variant parent capsid polypeptide. In some embodiments, the variant capsid polypeptide exhibits increased tropism as compared to a non-variant parent capsid polypeptide. In some embodiments, the variant capsid polypeptide further exhibits increased transduction or tropism in one or more non-liver or non-pancreatic human tissues as compared to a non-variant parent capsid polypeptide. In some embodiments, the variant capsid polypeptide exhibits increased transduction of human liver or pancreatic tissue or cells in vivo as compared to a non-variant parent capsid polypeptide. In some embodiments, the variant capsid polypeptide exhibits increased transduction of human liver or pancreatic tissue or cells in vitro as compared to a non-variant parent capsid polypeptide. In some embodiments, the variant capsid polypeptide exhibits increased transduction of human liver or pancreatic tissue or cells ex vivo as compared to a non-variant parent capsid polypeptide.

### EXAMPLES

### Example 1: DARPin Selection System for Fast and Reliable Selection of Specific Surface Receptor Targeted AAVs

High diversity DARPin libraries were incorporated into the AAV capsid via a VP2-fusion protein, thus generating either replication-deficient or -competent AAV library systems. This allows for direct screening using therapeutically relevant cell types in vitro and in vivo. The studies described herein show that replicating deficient and replicating AAV library can be generated with high diversity and functional titers while retaining their infectivity. A DARPin-capsid library was generated in AAV-DJ (a serotype with diverse cellular tropism) and AAV-2 (the most widely used serotype), and after several selection rounds, obtained strong enrichment for DARPin-capsids with high binding affinity for a specific cellular receptor, e.g., CD200 or ASGPR.

A goal in human gene therapy is the specific and exclusive modification of the desired target cells upon systemic vector administration. Vectors derived from adeno-associated viruses are among the most promising gene transfer systems for in vivo application. However, AAV specificity for a particular target cell or tissue has been hampered by the broad tropism of different AAV serotypes. Recent and new approaches have been initiated to create and select for more effective and selective AAV vectors by genetically modifying the capsid protein. These methods include random and/or rationale amino acid substitutions, creation of chimeric capsid variant libraries and selection screenings, and/or peptide insertion.

Described herein is a different approach for developing highly effective and selective AAV vectors that are specific to either pancreatic cells or liver cells. The method involves the incorporation of DARPins which are highly specific binding molecules into the AAV capsid. DARPins are derived from ankyrin-repeat proteins that have been developed as an alternative to antibody-based scaffolds, which are selected using high-throughput screening of DARPin libraries. Prior art DARPin selection systems can be difficult to perform and fail to yield the desired DARPin-AAV capsids. In some cases, only a low number of functional DARPin capsin chimeras were able to assemble into a function vector and bind their intended extracellular target.

The method described herein provides an improved system for directly selecting and obtaining DARPin-AAV capsids with desired binding properties. The AAV system can be a replicating AAV system or a non-replicating AAV system with a large insertion in the cap-frame.

### DARPin-AAV Non-Replicating Selection

Provided herein is a method for constructing a DARPin-nonreplicating AAV library system and selecting receptor-specific DARPins-AAV variants that can infect target cells. Described herein are DARPin-AAV variants that bind to CD200 that is expressed on pancreatic islet cells. Also described are DARPin-AAV variants that bind to ASGPR that is expressed on liver cells.

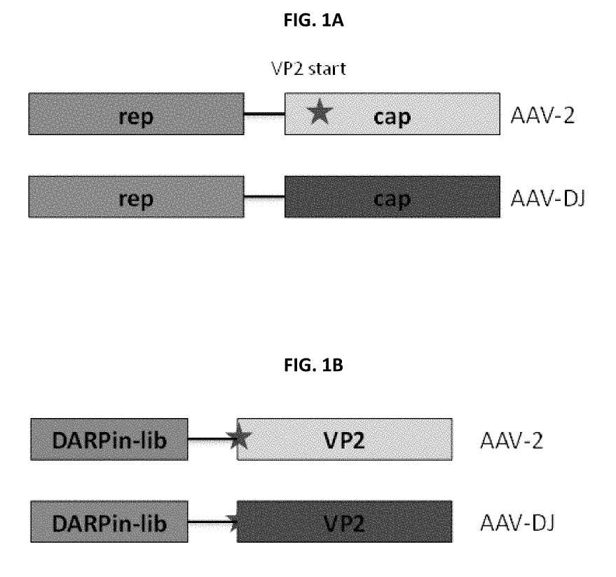

To construct DARPin-AAV libraries, open reading frames of AAV-2 and AAV-DJ cap were mutated at the VP2 start codon (FIG. 1A). Members of the DARPin N3C libraries were fused in-frame with respect to the VP2 reading frames that contain VP2 start site mutations (FIG. 1B).

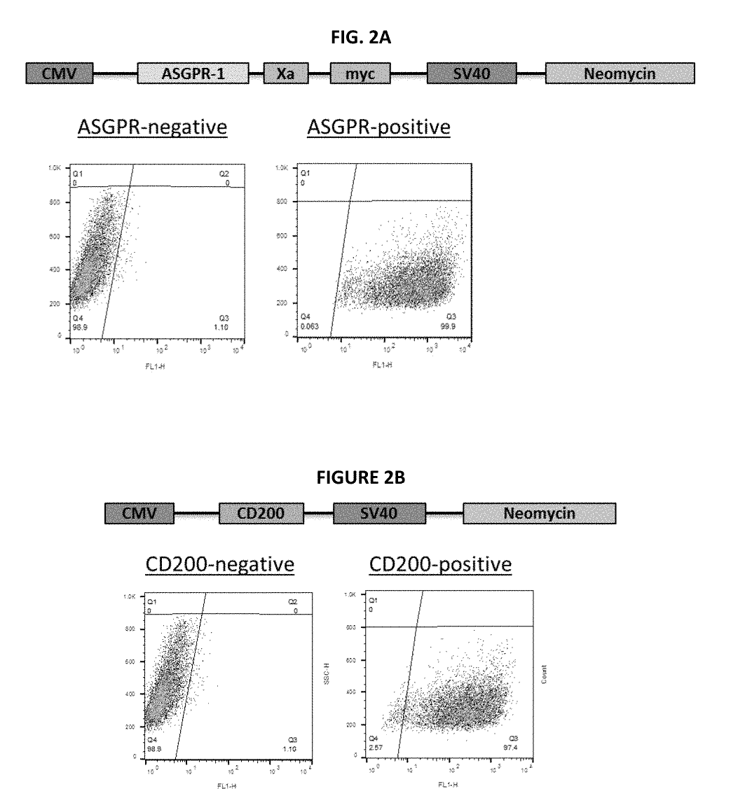

Cell lines for screening the DARPin-AAV library were generated by transfecting CHO-k1 cells with expression plasmids containing either the extracellular domain of mouse ASGPR-1 or human CD200 along with a neomycin selection cassette. The transfected cells underwent G418 selection and were FACS sorted for receptor positive cells. CD200-expressing cells were detected with an antibody that recognizes the receptor. ASGPR-expressing cells were detected using a myc-tag specific antibody. FIG. 2A depicts a schematic of the ASGPR expression construct which includes a myc tag and the FACS sorted transfected cells. FIG. 2B shows a schematic of the CD200 expression construct and the FACS sorted transfected cells.

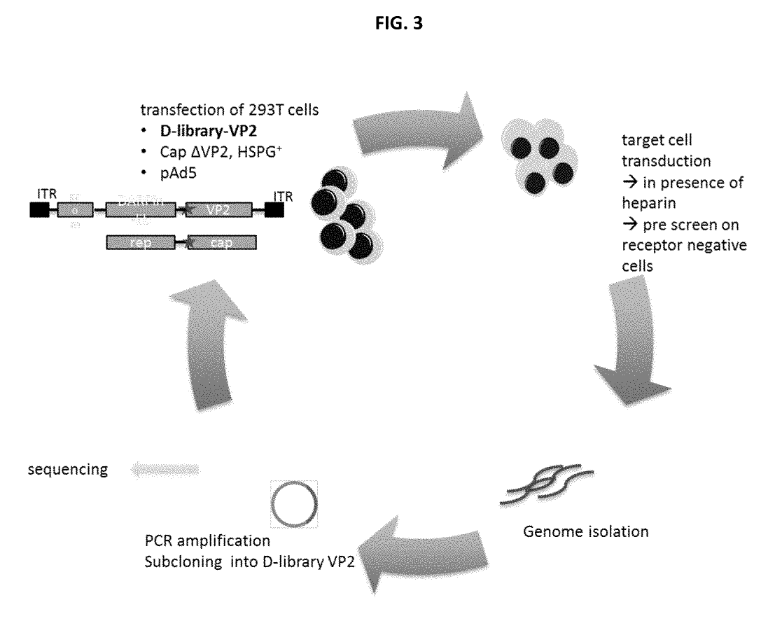

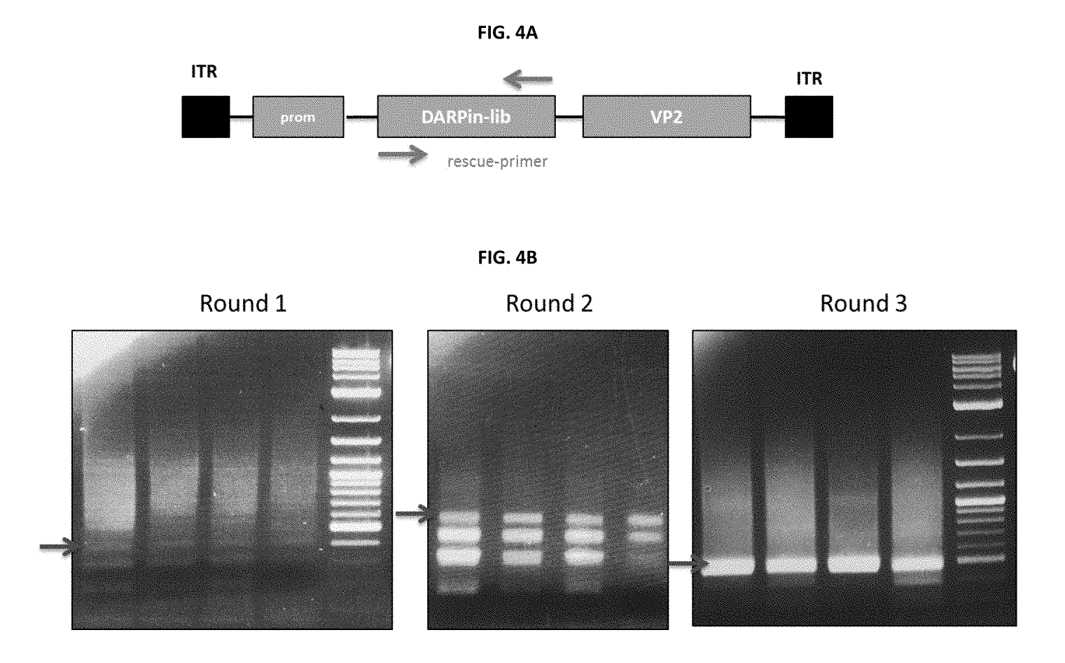

Viral particles of the DARPin-AAV libraries were produced by transient transfection of 293T cells with (a) the library plasmid cloned into an AAV vector containing ITR sequence, (b) a packaging plasmid, and (c) an adenoviral helper plasmid. Receptor-negative CHO-k1 cells were incubated with purified DARPin-AAV library viral stocks for 6-12 hours (pre-screening). The supernatant was subsequently transferred to CHO cells expressing the desired receptor (CD200 or ASGPR). After 24 hours, the cells were washed and genomic DNA was isolated. An amplicon containing a DARPin sequence was generated by PCR using rescue primers and subcloned into the library plasmid, initiating a new selection cycle. FIG. 3 provides a schematic diagram of the library selection process. After each round of selection, individual DARPin pools were isolated using rescue primers and subcloned into the library plasmid (FIG. 4A). Individually selected DARPin pools showed increasing signal intensity after each round of selection, indicating successful enrichment of selected DARPin pools (FIG. 4B).

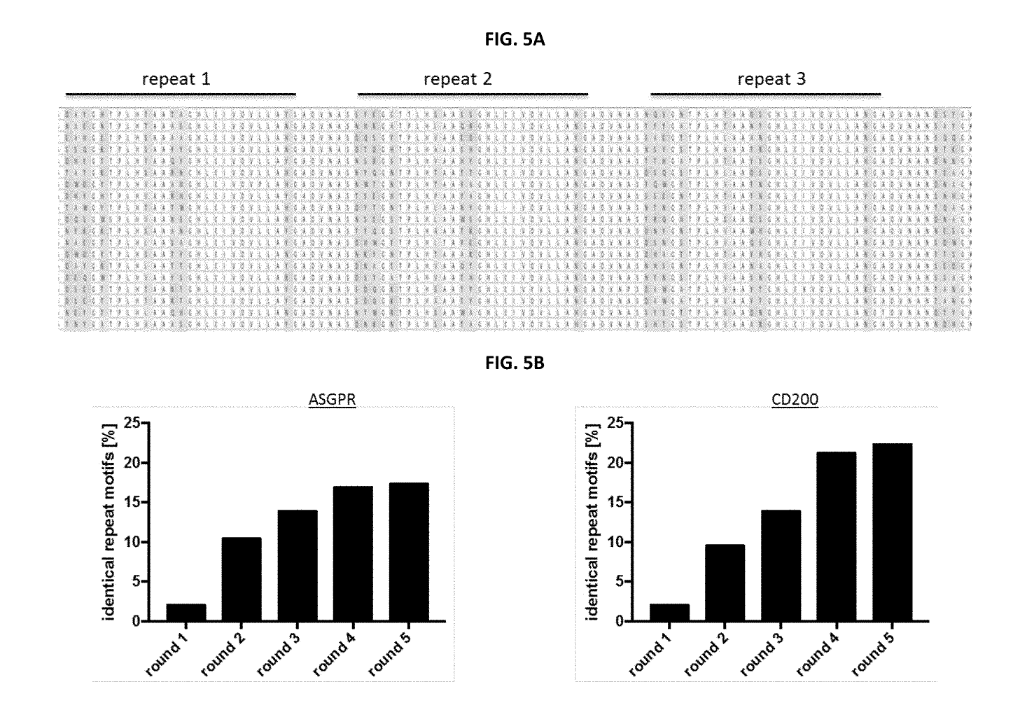

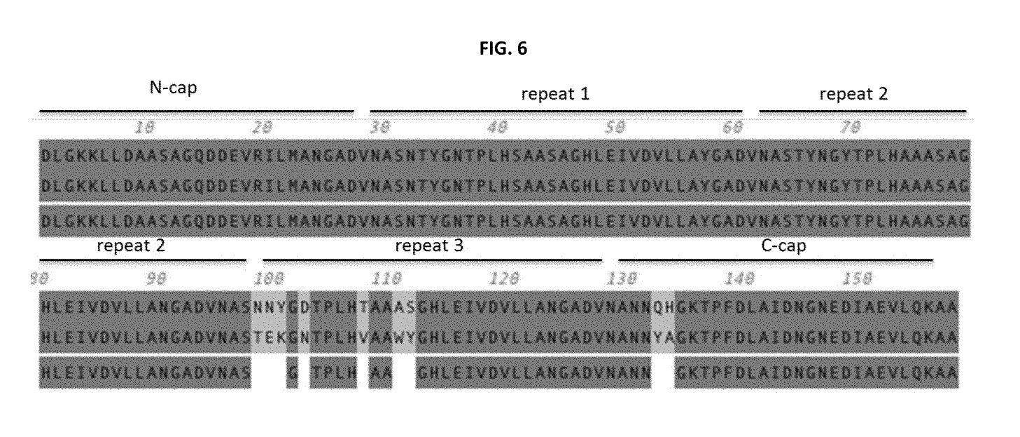

After each selection round and subsequent subcloning into the library plasmid, 100 clones from each pool were sequenced and aligned. Individual repeat motifs were analyzed to identify recurring sequence motifs (FIG. 5A). For both the ASGPR-specific DARPin pool and the CD200-specific DARPin pool, the number of identical repeat motifs increased from selection round 1-5, thus indicating successful selection of receptor-specific DARPins (FIG. 5B). Two CD200-specific DARPins (DARPin-CD200-19 and DARPin-CD200-23) were identified (FIG. 6). The amino acid sequences of these two DARPins were identical except for 7 amino acid residues in the C-terminal repeat motif and 2 amino acid residues in the C-cap.

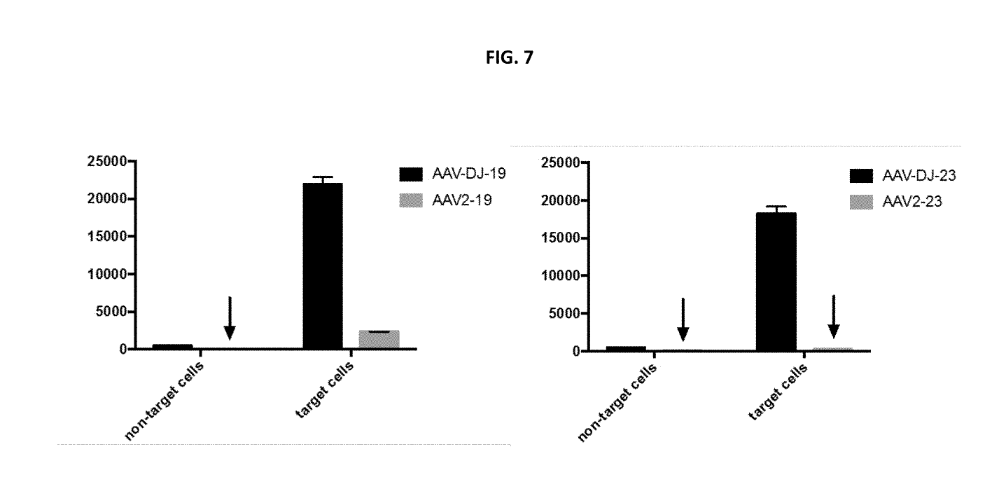

To determine if the infectivity and specificity of the CD200-specific DARPin-AAV vectors, they were used to transduce GFP into CD200 expressing cells (e.g., CHO-CD200 cells). FIG. 7 shows that DARPin-CD200-19-AAV-DJ and DARPin-CD200-23-AAV-DJ vector produced GFP expressing CHO-CD200 cells.

### DARPin-AAV Replicating Selection

Provided herein is a method for constructing a DARPin-replicating AAV library system and selecting receptor-specific DARPins-AAV variants that can infect target cells.

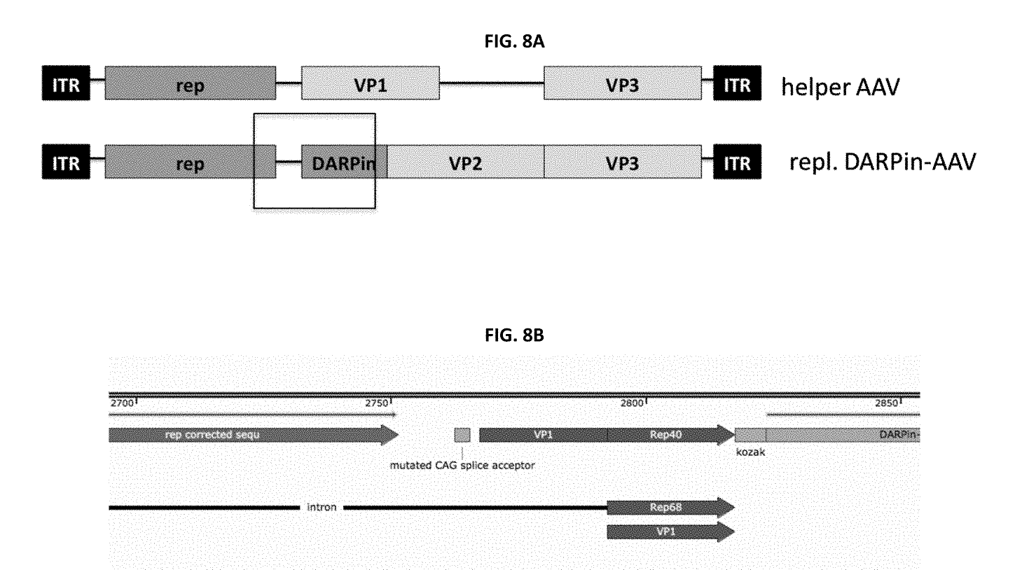

The helper AAV was constructed by mutation the AAV-2 start site, creating a self-replicating virus lacking a VP2 protein. The replicating DARPin-AAV was generated by fusing the DARPin sequence in-frame with the VP2 reading frame, thus deleting some sequences of the VP1 reading frame (FIGS. 8A and 8B).

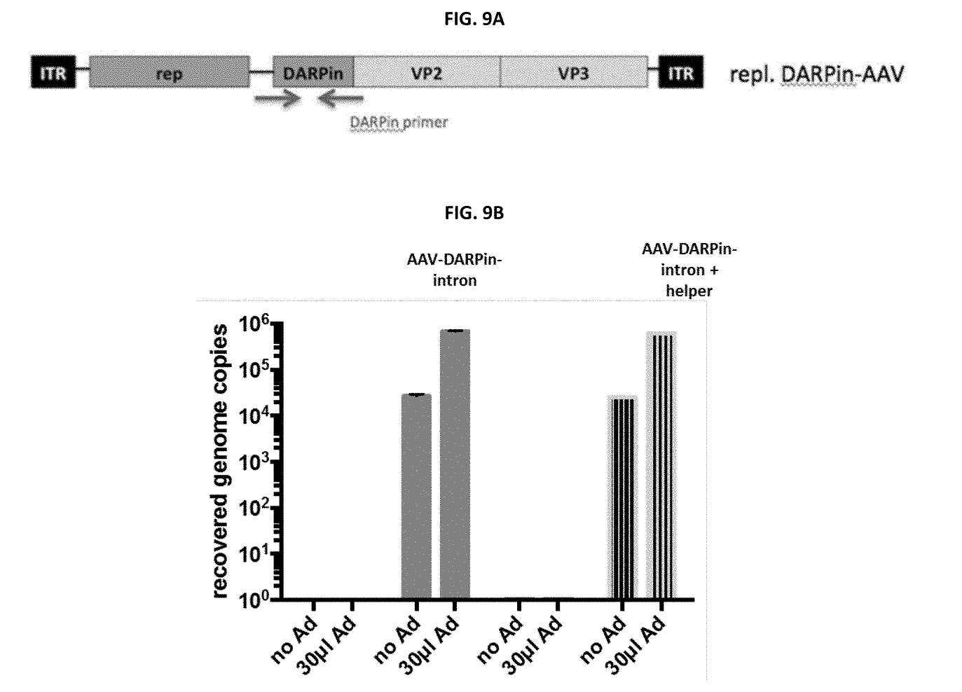

SK-OV-3 cells were transduced with the self-replicating DARPin-AAV and infected with adenovirus 6 hours after transduction. After 3 days, the resulting cells were harvested and genomic DNA was isolated. Replication was detected using primers shown in FIG. 9A. The presence of the DARPin was compared to samples obtained from cell that were not infected with adenovirus (FIG. 9B).

### Example 2: Selection of Next Generation AAV Gene Therapy Vectors for Specific and Precise Gene Delivery

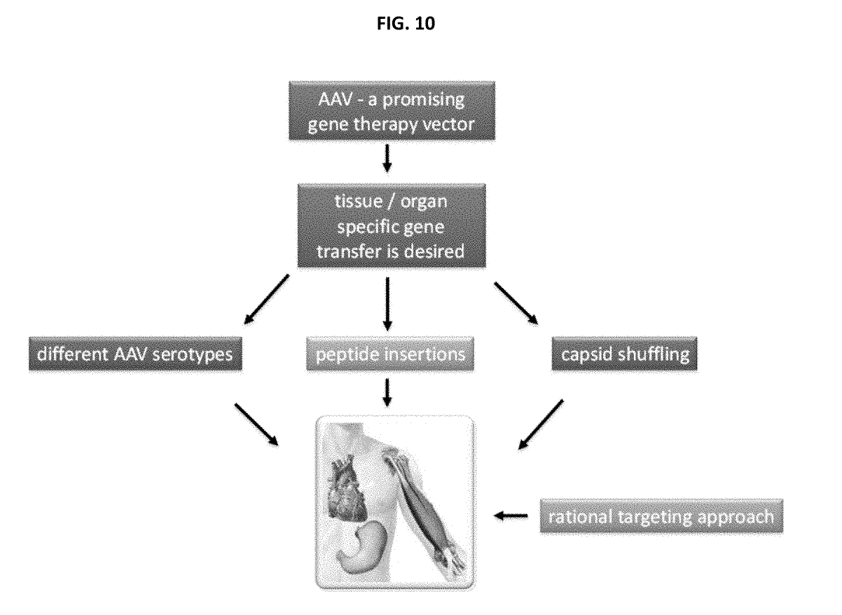

The ultimate goal in human gene therapy is the specific and exclusive modification of the desired target cells upon systemic vector administration (FIG. 10). Especially vectors derived from adeno-associated virus (AAV) are among the most promising gene transfer systems for in vivo application and have received broad attention due to substantial clinical benefit.

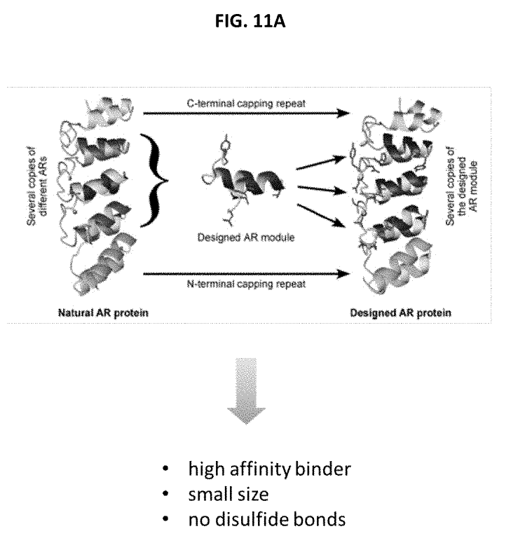

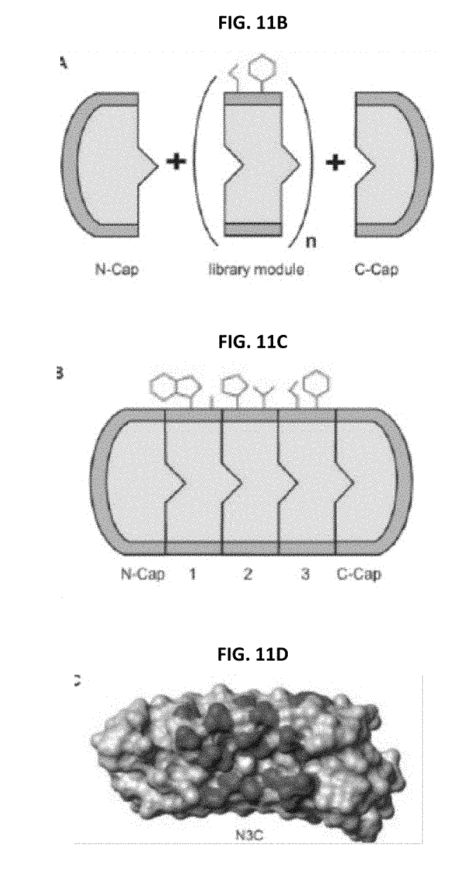

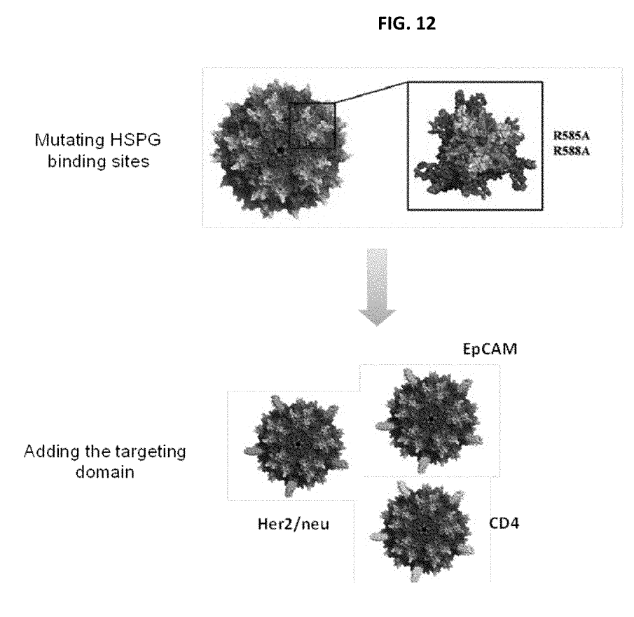

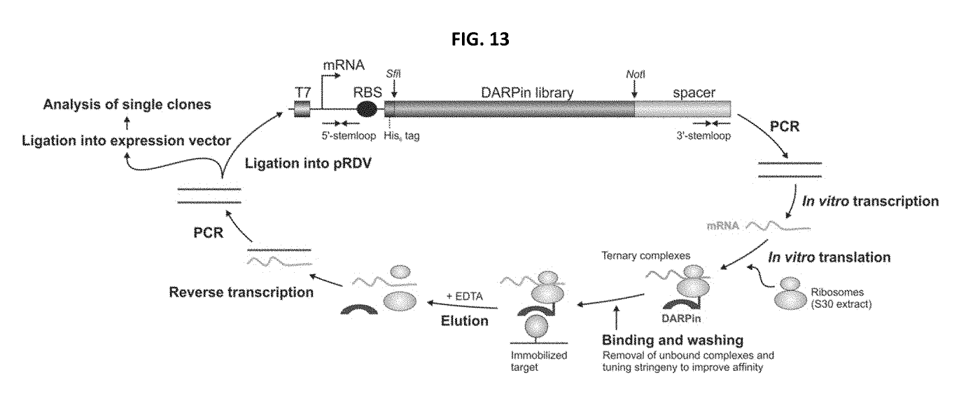

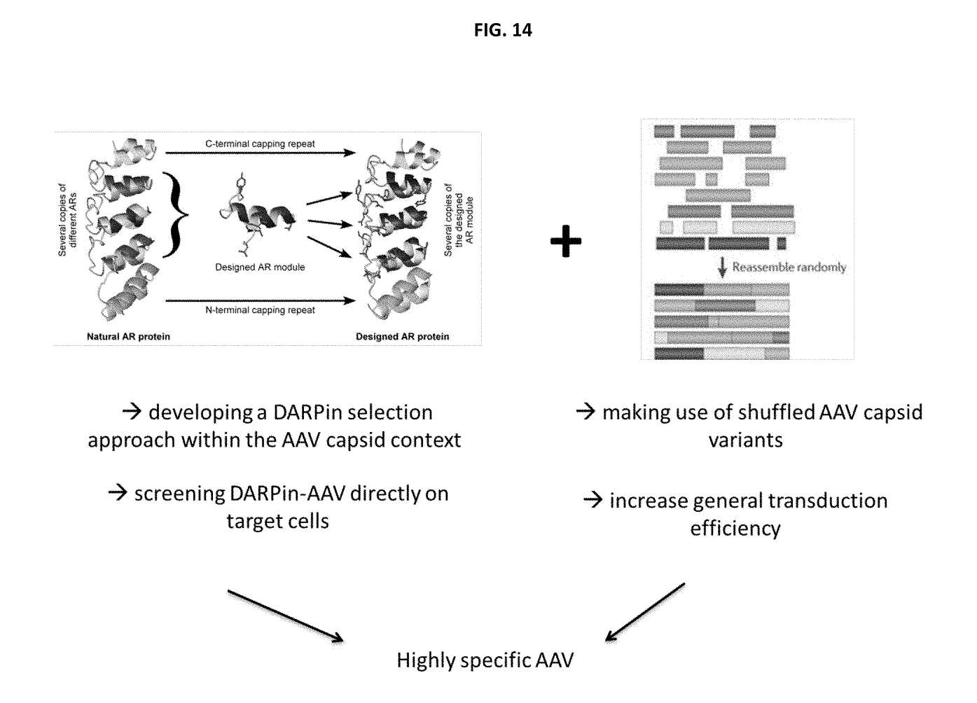

However, AAV specificity for a particular target cell or tissue has been hampered by the broad tropism of different AAV serotypes. Over the last several years, new approaches have been initiated to create and select for more effective and selective recombinant AAV vectors by genetically modifying the capsid protein. These methods include random and/or rationale amino acid substitutions, creation of chimeric capsid variant libraries and various selection screens, and/or peptide insertion. A different approach involves the incorporation of highly specific binding molecules (DARPins) into the AAV capsid (FIG. 12). DARPins are derived from ankyrin-repeat proteins that have been developed as alternative to antibody-based scaffolds, which are selected by high-throughput screens from DARPin libraries (FIG. 11A-FIG. 11D). At present, the bottleneck remains the cumbersome selection of DARPin molecules and the low number of functional DARPin capsid chimeras, which are able to assemble into a functional vector and still bind their intended extracellular target (FIG. 13). Thus after months of screening and analysis the majority of the pre-selected DARPins are found not to be suitable for AAV targeting approaches.

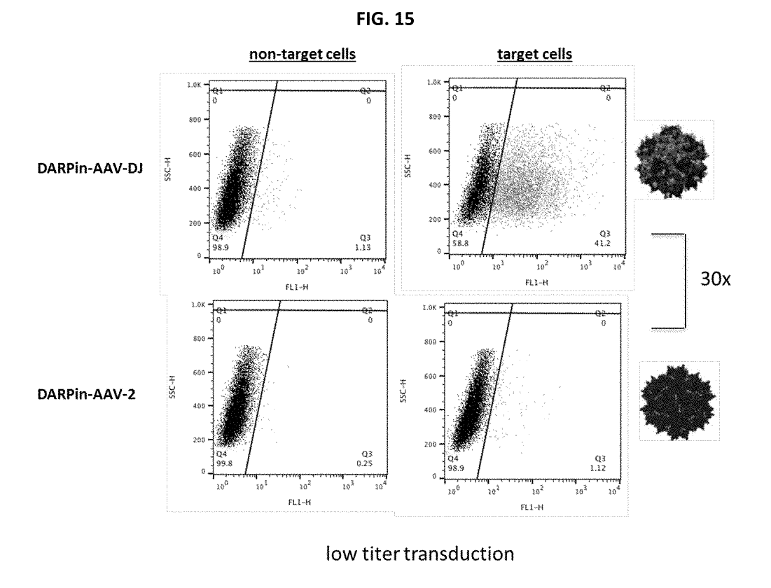

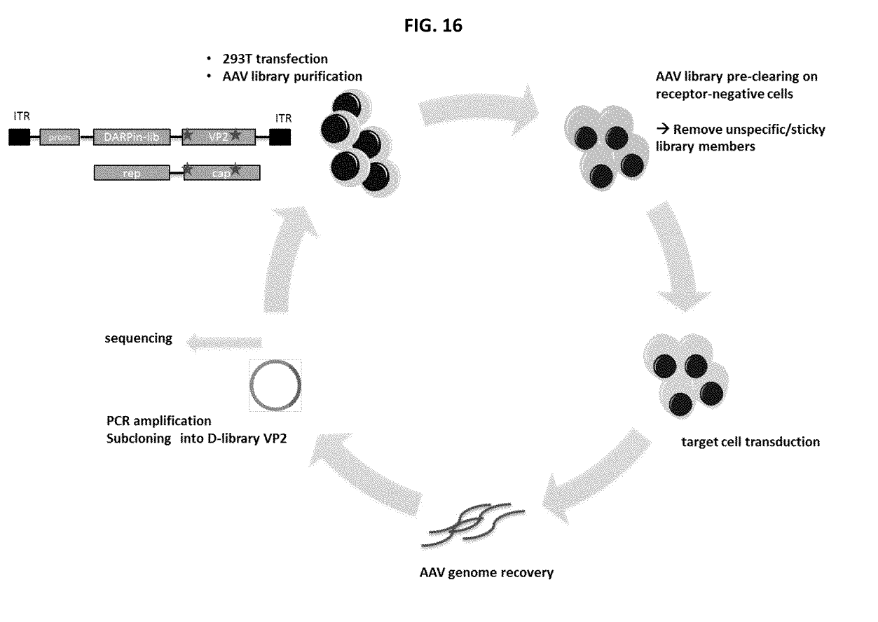

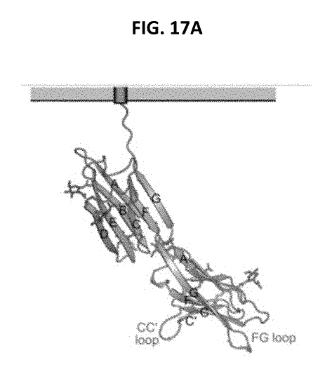

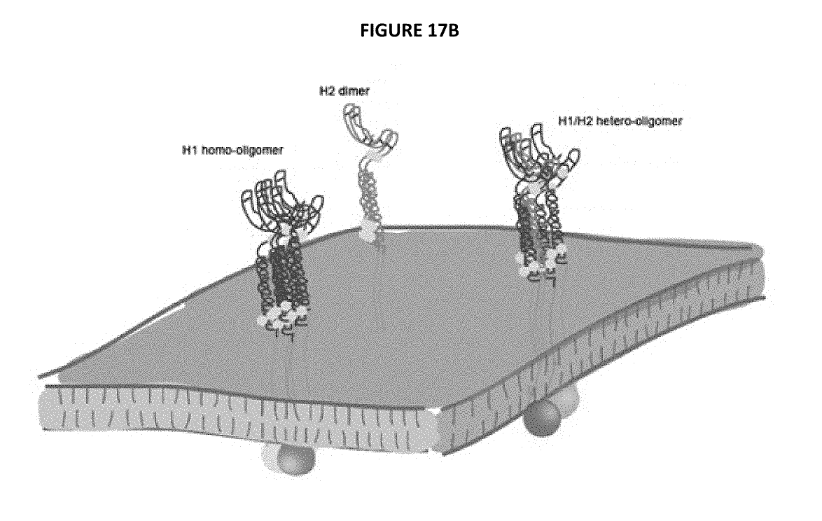

Described herein is a novel reliable and faster selection approach (FIG. 14). First the cumbersome and often misleading prokaryotic selection steps were eliminated. To do this, an entire DARPin library was incorporated into the AAV capsid as VP2-fusion protein, generating either replication deficient or competent AAV library systems allowing for direct screening on therapeutically relevant cell types in vitro or in vivo. The studies show that replicating deficient and replicating AAV libraries can be generated with high diversity up to 5×10⁷ and high functional titers retaining their infectivity. The studies have generated a DARPin-capsid library in AAV-DJ (serotype with diverse cellular tropism) and AAV-LK03 (human selective serotype) and after only two selection rounds, found up to 10% of selected DARPins sharing identical repeat motifs responsible for target receptor binding in the pancreas and the liver (FIG. 15). This new approach takes only six days per selection cycle, allowing discovery of novel DARPin molecules and selection for targeted vectors in substantially reduced time (FIG. 16). This approach expands the potential diversity in creating and defining novel rAAV vectors with medically relevant transduction properties (FIGS. 17A and 17B). Such viral vectors will not only open the door for an array of new approaches to treat acquired human diseases but also push the development of new AAV-based gene therapy vectors to the next level.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A variant adeno-associated virus (AAV) capsid polypeptide comprising a designed ankyrin repeat protein (DARPin) or fragment thereof fused to the N-terminus of an AAV capsid protein VP2,
wherein said DARPin specifically binds to a cell surface molecule expressed on human liver tissue or cells and said variant capsid polypeptide exhibits increased transduction or tropism in human liver tissue or cells as compared to a non-variant parent capsid polypeptide, or
wherein said DARPin specifically binds to a cell surface molecule expressed on human pancreatic tissue or cells and said variant capsid polypeptide exhibits increased transduction or tropism in human pancreatic tissue or cells as compared to a non-variant parent capsid polypeptide.

2. The variant AAV capsid polypeptide of claim 1, wherein said variant capsid polypeptide further exhibits an enhanced neutralization profile as compared to a non-variant parent capsid polypeptide.

3. The variant AAV capsid polypeptide of claim 1, wherein said variant capsid polypeptide further exhibits increased transduction or tropism in one or more non-liver human tissues or one or more non-pancreatic human tissues as compared to a non-variant parent capsid polypeptide.

4. The variant AAV capsid polypeptide of claim 1 wherein said cell surface molecule expressed on human liver tissue or cells is asialoglycoprotein receptor (ASGPR) or said cell surface molecule expressed on human pancreatic tissue or cells is CD200.

5. The variant AAV capsid polypeptide of claim 4, wherein said variant capsid polypeptide comprises an amino acid sequence having at least 85% sequence identity to the sequence selected from the group consisting of SEQ ID NOs: 1 to 4.

6. The variant AAV capsid polypeptide of claim 1, wherein said variant capsid polypeptide is part of a functional AAV capsid, wherein said functional AAV capsid packages a nucleic acid sequence selected from the group consisting of a non-coding RNA, a protein coding sequence, an expression cassette, a multi-expression cassette, a sequence for homologous recombination, a genomic gene targeting cassette, and a therapeutic expression cassette.

7. A method of using the variant AAV capsid polypeptide of claim 1 in a therapeutic treatment regimen or vaccine.

8. A method of using the variant AAV capsid polypeptide of claim 1 to reduce the amount of total nucleic acid administered to a subject, said method comprising administering less total nucleic acid amount to said subject when said nucleic acid is transduced using a variant capsid polypeptide as compared to the amount of nucleic acid administered to said subject when said nucleic acid is transduced using a non-variant parent capsid polypeptide in order to obtain a similar therapeutic effect.

9. An adeno-associated virus (AAV) vector comprising a nucleic acid sequence encoding a variant capsid polypeptide comprising a designed ankyrin repeat protein (DARPin) or fragment thereof fused to the N-terminus of an AAV capsid protein VP2,
wherein said DARPin specifically binds to a cell surface molecule expressed on human liver tissue or cells and said variant capsid polypeptide exhibits increased transduction or tropism in human liver tissue or cells as compared to a non-variant parent capsid polypeptide, or
wherein said DARPin specifically binds to a cell surface molecule expressed on human pancreatic tissue or cells and said variant capsid polypeptide exhibits increased transduction or tropism in human pancreatic tissue or cells as compared to a non-variant parent capsid polypeptide.

10. The AAV vector of claim 9, wherein said variant capsid polypeptide further exhibits an enhanced neutralization profile as compared to a non-variant parent capsid polypeptide.

11. The AAV vector of claim 9, wherein said variant capsid polypeptide further exhibits increased transduction or tropism in one or more non-liver human tissues or one or more non-pancreatic human tissues as compared to a non-variant parent capsid polypeptide.

12. The AAV vector of claim 9, wherein said cell surface molecule expressed on human liver tissue or cells is asialoglycoprotein receptor (ASGPR), or said cell surface molecule expressed on human pancreatic tissue or cells is CD200.

13. The AAV vector of claim 12, wherein said variant capsid polypeptide comprises an amino acid sequence having at least 85% sequence identity to the sequence selected from the group consisting of SEQ ID NOs: 1 to 4.

14. The AAV vector of claim 9, wherein said vector further comprises a nucleic acid sequence selected from the group consisting of a non-coding RNA, a coding sequence, an expression cassette, a multi-expression cassette, a sequence for homologous recombination, a genomic gene targeting cassette, and a therapeutic expression cassette.

15. A method of using the AAV vector of claim 9 in a therapeutic treatment regimen or vaccine.

16. A method of using the AAV vector of claim 9 to reduce the amount of total AAV vector administered to a subject, said method comprising administering less total AAV vector amount to said subject when said AAV vector is transduced by a variant capsid polypeptide as compared to the amount of AAV vector administered to said subject when said AAV vector is transduced by a non-variant parent capsid polypeptide in order to obtain a similar therapeutic effect.

17. A method for generating a variant AAV capsid polypeptide comprising a designed ankyrin repeat protein (DARPin) or fragment thereof fused to the N-terminus of an AAV capsid protein VP2, wherein said DARPin specifically binds to a cell surface molecule expressed on human liver or pancreatic tissue or cells, wherein said variant capsid polypeptide exhibits increased transduction or tropism in human liver or pancreatic tissue or cells as compared to a non-variant parent capsid polypeptide, said method comprising:
a) generating a library of variant capsid polypeptide genes, wherein said variant capsid polypeptide genes include a plurality of variant capsid polypeptide genes comprising sequences from more than one non-variant parent capsid polypeptide;
b) generating an AAV vector library by cloning said variant capsid polypeptide gene library into AAV vectors, wherein said AAV vectors are replication competent AAV vectors;
c) screening said AAV vector library from b) for variant AAV capsid polypeptides for increased transduction or tropism in human liver and pancreatic tissue or cells as compared to a non-variant parent capsid polypeptide; and
d) selecting said variant AAV capsid polypeptides from c).

18. The method of claim 17, wherein c) further comprises transducing target cells with said AAV vector library.

19. The method of claim 18, wherein said target cells are recombinant cells expressing a cell surface molecule expressed on human liver or pancreatic tissue or cells.

20. The method of claim 19, wherein said cell surface molecule expressed on human liver tissue or cells is ASGPR or said cell surface molecule expressed on human pancreatic tissue or cells is CD200.


SEQUENCE LISTING (13 sequences)

SEQ ID NO: 1 - DNA, 472 bp, Artificial Sequence
  Synthetic Nucleic Acid
gacctgggtaagaaactactggatgctgctagtgctggtcaggatgacgaagttcgtatactgatggctaacggtgctgacgttaacgcttctaacacttacggtaacactccgctgcactctgctgcttctgctggtcacctggaaatcgtcgatgttctgctggcttacggtgctgacgttaacgcttctacttacaacggttacactccgctgcacgctgctgcttctgctggtcacctggaaatcgtcgatgttctgctggctaacggcgctgacgttaacgcttctaacaactacggtgacactccgctgcacactgctgctgcttctggtcacctggaaatcgtcgatgttctgctggctaacggggctgacgtcaacgctaacaaccagcatggtaagaccccgttcgacttagcgatcgacaacggtaacgaggacattgctgaagtgctgcaaaaagcggcac

SEQ ID NO: 2 - DNA, 471 bp, Artificial Sequence
  Synthetic Nucleic Acid
gacctcggaaaaaaacttttggatgcggcgagtgccggtcaggatgatgaagttcggatccttatggcgaatggagctgatgtgaacgcaagcaacacttatgggaatacgcctttgcactcagcagcttccgccggacacctcgaaatcgttgatgtactgttggcatatggggcagatgtgaatgcatccacgtataacggctatacgcccctccatgctgcagcttcagcagggcatctcgagatcgtggatgtcctgctcgccaacggtgccgatgtgaacgccagcaacaactacggtgatactccgctgcacacggctgcagctagtggccacttggagatagtcgatgtcttgttggcgaatggggcggacgttaatgctaataaccaacacggaaaaacaccattcgatttggctatagataatggaaatgaagacatcgctgaagttttgcaaaaagccgca

SEQ ID NO: 3 - DNA, 472 bp, Artificial Sequence
  Synthetic Nucleic Acid
gacctgggtaagaaactgctggatgctgctagtgctggtcaggatgacgaagttcgtatactgatggctaacggtgctgacgttaacgcttctaacacttacggtaacactccgctgcactctgctgcttctgctggtcacctggaaatcgtcgatgttctgctggcttacggtgctgacgttaacgcttctacttacaacggttacactccgctgcacgctgctgcttctgctggtcacctggaaatcgtcgatgttctgctggctaacggcgctgacgttaacgcttctactgaaaaaggtaacactccgctgcacgttgctgcttggtacggtcacctggaaatcgtcgatgttctgctggctaacggggctgacgtcaacgctaacaactacgctggtaagaccccgttcgacttagcgatcgacaacggtaacgaggacattgctgaagtgctacaaaaagcggcac

SEQ ID NO: 4 - DNA, 471 bp, Artificial Sequence
  Synthetic Nucleic Acid
gaccttgggaagaagctgcttgatgctgcgtccgccggacaggatgatgaagttcgcatccttatggctaatggcgcggacgtgaatgcatccaacacgtatggcaacactcccttgcactctgctgcctctgccgggcatcttgagatagtggacgtgttgttggcatatggtgccgatgtaaatgcttcaacttacaacggatacacaccgcttcatgctgcggcatcagcgggtcatcttgagatagtagacgtgctgctggctaacggggcggatgtgaacgcgagcacagaaaagggaaatactccacttcacgtggcagcgtggtacggtcatttggaaattgttgacgtcctcctggcgaatggtgcggacgtaaatgccaataattacgcgggtaagacaccgttcgaccttgccatcgacaacggaaatgaagacattgcggaggttcttcaaaaagcagca

SEQ ID NO: 5 - protein, 735 aa, Artificial Sequence
  Synthetic Polypeptide
MAADGYLPDWLEDNLSEGIREWWALKPGVPQPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYNKSVNVDFTVDTNGVYSEPRPIGTRYLTRNL

SEQ ID NO: 6 - protein, 735 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 7 - DNA, 2208 bp, Artificial Sequence
  Synthetic Nucleic Acid
atggctgccgatggttatcttccagattggctcgaggacaacctttctgaaggcattcgtgagtggtgggctctgaaacctggagtccctcaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaatgatggcaccaagaccatcgccaataacctcaccagcaccatccaggtgtttacggactcggagtaccagctgccgtacgttctcggctctgcccaccagggctgcctgcctccgttcccggcggacgtgttcatgattccgcagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgctcacagccagagtctggaccgtctcatgaatcctctcatcgaccagtacctgtattacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccgtctccgctgatgggcggctttggcctgaaacatcctccgcctcagatcctgatcaagaacacgcctgtacctgcggatcctccgaccaccttcaaccagtcaaagctgaactctttcatcactcagtattctactggccaagtcagcgtggagatcgagtgggagctgcagaaggaaaacagcaagcgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 2208 bp, Artificial Sequence
  Synthetic Nucleic Acid
atggctgccgatggttatcttccagattggctcgaggacaaccttagtgaaggaattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccgtctccgctgatgggcggctttggcctgaaacatcctccgcctcagatcctgatcaagaacacgcctgtacctgcggatcctccgaccaccttcaaccagtcaaagctgaactctttcatcactcagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 9 - protein, 735 aa, Artificial Sequence
  Synthetic Polypeptide
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTIQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - DNA, 2208 bp, Artificial Sequence
  Synthetic Nucleic Acid
atggctgccgatggttatcttccagattggctcgaggacaaccttagtgaaggaattcgcgagtggtgggctttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattcccaatggctgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcaccatccaggtgtttacggactcggagtaccagctgccgtacgttctcggctctgcccaccagggctgcctgcctccgttcccggcggacgtgttcatgattccgcaatacggctacctgacgctcaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttggaaagcaaggctcagagaaaacaaatgtggacattgaaaaggttatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtagacactaatggtgtttatagtgaacctcgccctattggaacccggtatctcacacgaaacttgtaa

SEQ ID NO: 11 - protein, 156 aa, Artificial Sequence
  Synthetic Polypeptide
DLGKKLLDAASAGQDDEVRILMANGADVNASNTYGNTPLHSAASAGHLEIVDVLLAYGADVNASTYNGYPLHAAASAGHLEIVDVLLANGADVNASNNYGDTPLHTAAASGHLEIVDVLLANGADVNANNQHGKTPFDLAIDNGNEDIAEVLQKAA

SEQ ID NO: 12 - protein, 156 aa, Artificial Sequence
  Synthetic Polypeptide
DLGKKLLDAASAGQDDEVRILMANGADVNASNTYGNTPLHSAASAGHLEIVDVLLAYGADVNASTYNGYPLHAAASAGHLEIVDVLLANGADVNASTEKGNTPLHVAAWYGHLEIVDVLLANGADVNANNYAGKTPFDLAIDNGNEDIAEVLQKAA

SEQ ID NO: 13 - protein, 147 aa, Artificial Sequence
  Synthetic Polypeptide
DLGKKLLDAASAGQDDEVRILMANGADVNASNTYGNTPLHSAASAGHLEIVDVLLAYGADVNASTYNGYPLHAAASAGHLEIVDVLLANGADVNASGTPLHAAGHLEIVDVLLANGADVNANNGKTPFDLAIDNGNEDIAEVLQKAA